(19) United States
(12) Patent Application Publication
(10) Pub. No.: US 2004/0028651 A1
(43) Pub. Date: Feb. 12, 2004

(54) Composition and methods of using hiv vpr

(76) Inventors: Karrupiah Muthumani, Upper Darby, PA (US); David B. Weiner, Merion Station, PA (US)

Correspondence Address: COZEN O'CONNOR, P.C., 1900 MARKET STREET, PHILADELPHIA, PA, 19103-3508, US

(21) Appl. No.: 10/311,260

(22) PCT Filed: Mar. 29, 2001
(86) PCT No.: PCT/US01/10028

(51) Int. Cl. A61K 48/00

(52) U.S. Cl. 424/93.2; 514/44

(57) ABSTRACT

Methods of delivering a desired polypeptide to an individual are disclosed. The methods comprise administering to the individual an immunogenic vector comprising a nucleic acid encoding the desired polypeptide operably linked to regulatory elements in combination with one or more of Vpr protein, a functional fragment of Vpr protein, a nucleic acid encoding Vpr protein operably linked to regulatory elements, or a nucleic acid encoding fragment of Vpr protein operably linked to regulatory elements. Methods of inhibiting an undesirable immune response in an individual are disclosed. Methods for inhibiting the cellular proliferation of a tumor cell in an individual are disclosed.

### CROSS-REFERENCE TO RELATED APPLICATIONS

This application claims priority to U.S. Provisional Patent Applications, Serial No. 60/193,495, filed Mar. 31, 2000, and Serial No. 60/231,141, filed Sep. 8, 2000, each of which is incorporated herein by reference.

### FIELD OF THE INVENTION

The present invention relates to methods of inhibiting anti-vital response, to improved gene delivery methods, to methods of treating individuals who have autoimmune diseases and to methods of treating individuals who have diseases, disorders and conditions associated with inflamation. The present invention relates to pharmaceutical compositions useful in such methods. The present invention relates to improved gene therapy vectors and compositions which comprise HIV Vpr or a gene encoding the same, and methods of making and using the same. The present invention relates to methods for delivering polypeptides to individuals while inhibiting the cellular immune response against the vector which contains the nucleic acid encoding the desired polypeptide.

### BACKGROUND OF THE INVENTION

One promise of gene therapy is the ability to correct genetic defects responsible for disease by the addition to an individual of fictional genetic material as well as the ability to deliver therapeutic proteins using genetic material that encodes such proteins. There is a great deal of activity in the development of protocols for treating diseases and disorders by administering a nucleic acid which codes for a polypeptide that is either missing or defective in an individual Another promise of gene therapy is as an alternative and improved means to deliver therapeutically important proteins to individuals in need of such proteins. The discovery of proteins with the therapeutically important functions has led to new treatments for many diseases and disorders and the application of gene therapy to deliver such proteins is also the subject of much interest.

Among the strategies for delivering genetic material, the use of immunogenic vectors, most commonly viral vectors, capable of infecting the individual's cells is one of the most widely employed methodologies. Essentially, genetic material that encodes desired proteins, whether they be functional forms of defective genes responsible for disease or coding sequences for therapeutically useful proteins, is incorporated into the genome of a vector which has the ability to infect cells of the individual or otherwise deliver the genetic material to cells of the individual.

Adenovirus, adeno-associated virus (AAV), vaccinia virus, and simian virus 40 (SV40) are just a few of the many viruses used to make viral vectors for gene therapy. In some cases, the viral vectors are selected for their ability to infect specific tissue to which delivery of the genetic material is desired. In some cases, the viral vectors are selected because they are attenuated and cause serious limited infections to the individual without significant pathology.

One of the major problems associated with gene therapy protocols that employ immunogenic vectors is that an immune response against the vector is induced in the individual who is administered the vector. The immune response targets the vector including cells which are infected by the vector. The destruction of cells which are infected by the vector reduces the efficacy of the treatment. Further, immune responses induced against the vectors limit the effectiveness of subsequent doses of the same gene therapeutic composition or other gene therapeutic compositions which use the same vector because the immune system of the individual will recognize the vector from the subsequent doses of the same gene therapeutic composition or other gene therapeutic compositions which use the same vector and mount an immune response similar to the manner in which a vaccine protects the individual from subsequent exposure to a pathogen.

There are two branches to the immune system. The humoral branch of the immune system involves antibodies which are secreted by B lymphoid cells and recognize specific antigens. Binding of antibodies to specific antigens inactivates the antigen. Antibodies may also bind to the antigen and activate other immune cells which destroy the bound antigen.

The cellular branch of the immune system involves specific cell types which recognize and destroy cells which display “foreign” antigens. Cytotoxic T cells (also referred to as cyotoxic T lymphocytes or CTLs) are an example of cells in the cellular branch of the immune systems CTLs recognize fragments of peptides which are displayed on the plasma membrane surface bound to major histocompatatibility complex (MHC) molecules. Cells tat display a peptide which is “foreign” elicit a cellular immune response. Cytotoxic T cells then destroy the cell displaying the foreign peptide fragments.

The HIV-1 accessory gene vpr encodes viral protein R (Vpr) which has been implicated in the regulation of many host cellular events including proliferation, differentation, apoptosis, cytokine production, and NF-κB mediated transcription (Levy et at., 1993, Cell, 72:541-550; Ayyavoo et al., 1997, Nat. Med., 3:1117-1123; Stewart et al., 1997, J. Virol., 71:5579-5592, each of which is incorporated herein by reference). NF-κB activation is important for the induction of some cytokines and chemokines which specifically expand antigen specific immune responses. NF-κB activation also plays an important role in the induction of proinflammatory cytokines, in particular tumor necrosis factor-alpha (TNFα), triggered through the CD28 costimulatory pathway (Moriuchi et at., 1997, J. Immunol., 158:3483-3491; Fraser et al., 1992, Mol. Cell. Biol., 12:4357-4363, each of which is incorporated herein by reference). The pattern of cytokine expression influences the nature and persistence of the inflammatory response. For instance, production of interferon-gamma (IFN-γ) and TNF are well-suited to induce enhanced cellular immunity, while interleukin-4 (IL-4) and IL-10 are associated with helping B cells develop into antibody-producing cells (Paul et al., 1994, Cell, 76:241-251, which is incorporated herein by reference). Studies using mutant NF-κB binding sites and Ikbα competition have shown that transcription factors including NF-κB and SP-1 are important for RANTES (Regulated on Activation, Normal T Expressed and Secreted) gene expression (Moriuchi et al., 1997, J. Immunol., 158:3483-3491, which is incorporated herein by reference).

CD8⁺ T cells are believed to play an important role in controlling HIV infection through CTL induction. Additionally, CD8⁺ T cells are involved in secretion of several actors including the β-chemokines RANTES, MIP-1α, MIP-1β, and MDC (Brinchman et al., 1990, J. Immunol., 114:2961-2966; Cocchi et al., 1996, Science, 270:1811-1815; Pal et al., 1997, Science, 278:695-698, each of which is incorporated herein by reference). CD8⁺ CTL is an important immunological defense against viral infections.

Chemokines are important for the regulation of lymphocyte recruitment in infection and immune activation (Schall et al., 1994, Curr. Opin. Immunol., 6:865-873, which is incorporated herein by reference). T cell activation results in synthesis of certain chemokines/cytokines which are necessary for antigen specific T helper cell as well as for cytotoxic effector cell expansion (Weiss et al., 1994, Cell, 76:263-274, which is incorporated herein by reference). In addition to their role in T cell trafficking and immune activation, the β-chemokines can inhibit HIV-1 infection in established macrophage cell lines as well as in primary lymphocytes through interference with viral coreceptors required for entry (Feng et al., 1996, Science, 272:872-877; Dornaz et al., 1996, Cell, 85:1149-1158, each of which is incorporated herein by reference). For example, chemokines are produced by some subsets of T cells following T cell receptor (TCR) and CD28/CTLA-4 co-ligation (Taub et al., 1996, J. Immunol., 156:2095-2103; Herold et al., 1997, J. Immmol., 159:4150-4153, each of which is incorporated herein by reference).

Induction of T cell proliferation, CTL activation and cytokine secretion require both the engagement of the TCR complex and interaction of either CD28/CTLA-4 costimulatory molecules with their ligands CD80 or CD86 present on antigen presenting cells (APCs), or CD40 with the CD40 ligand, to provide the necessary second signal (Fraser et al., 1994, Immunol. Today, 14:357-362; Crabtree et al., 1989, Science, 243:355-361; Linsley et al., 1993, An. Rev. Immunol., 11:191-212; June et al., 1994, Immunol. Today, 15:321-331, each of which is incorporated herein by reference). Studies have shown that T cell activation through CD28 enhances production of β-chemokines, yielding anti-viral effects (Carroll et al., 1997, Science, 276:273-276; Levine et al., 1996, Science, 272:1939-1943; Bisset et al., 1997, AIDS, 11:485-491, each of which is incorporated herein by reference). Recruitment and activation of CD8⁺ cells at the site of inflammation increases specific CTL precursor frequency (Stevenson et al., 1997, Eur. J. Immunol., 27:3259-3268; Doherty et al., 1997, Immunol. Reviews, 159:105-117, each of which is incorporated herein by reference). Blocking the synthesis of chemokines may ameliorate the symptoms of inflammatory disease which rely on the synthesis of chemokines to recruit cells responsible for the immune response.

Cellular immunity, specifically the MHC-restricted CTL response, is thought to play an intrinsic role in protection and clearance of a number of viral infections. Reduction in the number of CD8⁺ T cells in HIV-1 infected individuals has been correlated with reduced anti-viral effect and disease progression in parallel with the deterioration of the immune system (Mackewicz et al., 1991, J. Clin. Invest, 87:1462-1466; Pantaleo et al., 1997, Proc. Natl. Acad Sci. USA, 94:9848-9853, each of which is incorporated herein by reference). Information from studies on HIV-1 infected long-term non-progressors, uninfected infants born to HIV-1 infected mothers, and seronegative individuals repeatedly exposed but as yet uninfected have supported the role of CTL responses in controlling viral load and peas even disease progression (Borrow et al., 1994, J. Virol., 68:6103-6110; Rowland-Jones et al., 1995, Nat. Med., 1:59-94, each of which is incorporated herein by reference).

Overexpression and overproduction of cytokines is associated with a number of disease conditions. Cytokine overproduction is particularly important to the pathogenesis of bacterial septic shock, which is a condition that can develop within a few hours following infection by certain gram-negative bacteria, including, but not limited to, E. coli, Klebsiell pneumonia, Pseudomonas aeruginosa, Enterobacter aerogenes, Neisseria meningitidis, and various species of Salmonella, as well as some gram-positive bacteria. Septic shock can occur following sepsis, a condition in which pathogenic microorganisms or their toxins are present in the blood or in other tissues during infection or contamination. The symptoms of bacterial septic shock, which is often fatal, include a drop in blood pressure, fever, diarrhea, and widespread blood clotting in various organs. This condition afflicts about 500,000 individuals in the U.S. annually, and kills more than 20,000 in the U.S. and one million people world wide each year. The annual cost for treating bacterial septic shock is estimated at $5-10 billion. The septic shock develops when bacterial cell wall endotoxins stimulate macrophages to over produce IL-1 and TNFα; it is the increased levels of IL-1 and TNFα that cause the septic shock symptoms. The rapid onset of the condition and the high mortality rate call for methods of prophylactic treatment, particularly in situations where patients will be put at risk for septic shock, such as during and following surgery. For a review of systemic inflammatory response syndrome, sepsis, and septic shock, see Paterson & Webster, 2000, J. P Coll. Surg. Edinb., 45:178-182, which is incorporated herein by reference.

Toxic shock is a disease having similar characteristics to septic shock, that is triggered by toxins, produced by a variety of organisms, including bacteria, that act as superantigens. Superantigens bind simultaneously to a class II MHC molecule and to the Vβ domain of the T cell receptor, resulting in the activation of all T cells bearing a particular Vβ domain. Rather than being internalized, processed, and presented by antigen presenting cells, as conventional antigens are, superantigens bind directly to class II MHC molecules (see Herman et al., 1991, Ann. Rev. Immunol., 9:745-772, which is incorporated herein by reference). Due to this unique binding ability, superantigens can activate large numbers of T cells regardless of their antigenic specificity. A number of bacterial superantigens have been implicated as the causative agent of several diseases such as bacterial toxic shock and food poisoning. These include staphylococcal enterotoxins (among which staphylococcal enterotoxin B (SEB) is most prevalant), exfoliating toxins, and toxic-shock syndrome toxin (TSST1); streptococcal pyrogenic exotoxins SPEA and SPEC; and Mycoplasma arthritidus supernatant (MAS). The large number of T cells activated by these superantigens results in excessive production of cytokines derived from T helper type 1 (Th1) cells, which include IL-2, IFN-γ, and TNFβ. TSST1, for example, induces extremely high levels of TNF and IL-1. The resultant systemic reactions are similar to bacterial septic shock, including fever, widespread blood clotting, and shock. For reviews of the role of superantigens in immunological disease, see Murray et al, 1995, Am. Soc. Microbiol. News, 61:229-235, and Fraser et al., 2000, Mol. Med. Today, 6:125-32, each of which is incorporated herein by reference.

There remains a need for improved gene therapy vectors, compositions, and methods which can be used to increase the safety and efficacy of gene therapy technology. There remains a need for improved gene therapy vectors, compositions, and methods which can reduce or eliminate the immune response against the viral vector which limits the ability to expose the individual to subsequent doses of the therapeutic vector, other therapeutics, or vaccines employing the same vector. There is a need for methods for delivering polypeptides to individuals while inhibiting the cellular immune response against the vector which encodes the desired polypeptide. There is a further need for methods for modulating immune responses associated with inflammatory and autoimmune diseases and disorders, and for therapeutic and prophylactic treatment of septic shock, toxic shock and related diseases. There is also a need for improved methods of arresting the growth of hyperproliferating cells associated with such diseases as cancer.

### SUMMARY OF THE INVENTION

The present invention relates to methods of delivering a desired polypeptide to an individual. The methods comprise administering to the individual an immunogenic vector comprising a nucleic acid encoding the desired polypeptide operably linked to regulatory elements in combination with one or more of Vpr protein, a functional fragment of Vpr protein, a nucleic acid encoding Vpr protein operably linked to regulatory elements, or a nucleic acid encoding a functional fragment of Vpr protein operably linked to regulatory elements.

The present invention relates to compositions comprising an immunogenic vector that comprises a nucleic acid encoding the desired polypeptide operably linked to regulatory elements; and one or more of Vpr protein, a functional fragment of Vpr protein, a nucleic acid encoding Vpr protein operably linked to regulatory elements, or a nucleic acid encoding a functional fragment of Vpr protein operably linked to regulatory elements.

The present invention relates to methods for inhibiting an undesirable immune response in an individual. The methods comprise administering to the individual in an amount sufficient to inhibit an undesirable immune response one or more of Vpr protein, a functional fragment of Vpr protein, a nucleic acid encoding Vpr protein operably linked to regulatory elements, or a nucleic acid encoding a functional fragment of Vpr protein operably linked to regulatory elements.

The present invention relates to methods for inhibiting cellular proliferation of tumor cells in an individual. The methods comprise administering the individual, in an amount sufficient to inhibit cellular proliferation, a recombinant adenovirus comprising a nucleic acid encoding Vpr protein operably linked to regulatory elements or a nucleic acid encoding an anti-tumor fragment of Vpr protein operably linked to regulatory elements.

### BRIEF DESCRIPTION OF THE DRAWINGS

FIGS. 1A, 1B, 1C, 1D, 1E and 1F, which contain data from Example 1, show the effect of treatment with Vpr on cell subsets.

FIGS. 2A, 2B, 2C, 2D and 2E, which contain data from Example 1, show the effect of treatment with Vpr on PBMCs.

FIG. 3, which contains data from Example 1, depicts MIP-1α production in splenocytes from mice immunized with pNef with or without co-immunization of pVpr.

FIG. 4 shows data from immunohistochemical analysis of lymphocyte infiltration at the site of antigen expression as set forth in Example 2.

FIGS. 5A, 5B, 5C and 5D show cytotoxic T lymphocyte response induced by pNef or pGag-Pol in the presence or absence of pVpr co-immunization from experiments set forth in Example 2.

FIGS. 6A and 6B show cytokine production in splenocytes obtained from mice co-immunized with pNef in the presence or absence of pVpr from experiments set forth in Example 2.

FIGS. 7A and 7B show data from experiments in Example 2 analyzing the effect of pVpr on humoral responses generated by different antigens.

FIGS. 8A, 8B, 8C and 8D show data from experiments in Example 2 analyzing the effect of Vpr as a virion-associated molecule on expression of costimulatory molecules on antigen presenting cells.

FIG. 9 is a schematic depiction of the construction of the HIV vpr-containing adenoviral vector pAdCMV-vpr.

FIG. 10 is a schematic depiction of the generation of Adeno-vpr recombinant viral particles.

FIG. 11 presents an immunoblot of Vpr protein as produced in a baculovirus expression system (lane 1), and as expressed in Adeno-vpr infected cells (lane 2, labeled as “Adeno-Vpr (+)”). Lane 3 (labeled “Adeno-Vpr (−)”) presents the negative control protein extract from Adeno-lacZ infected cells.

FIG. 12 presents an immunofluorescence photograph showing Vpr expression (red fluorescence) in a human macrophage cell that has been infected with Adeno-vpr recombinant adenovirus.

FIG. 13 presents cell cycle analyses of HeLa cells that have been mock infected, infected with Adeno-lacZ viral particles, or infected with Adeno-vpr virus particles.

FIG. 14 presents immunofluorescence data reflecting the level of expression of macrophage activation markers CD80 and CD80 in human macrophages that have been mock infected, infected with Adeno-lacZ viral particles, or infected with Adeno-vpr virus particles. The values presented in association with each histogram indicate the percentage of the cells in the sample that are positive for the indicated marker. The vertical axis presents the number of events or cells; the horizontal axis is the intensity of fluorescence.

FIG. 15 presents data on the levels of chemokine production by human macrophages that have been mock infected, infected with Adeno-pr viral particles, or infected with Adeno-lacZ virus particles.

FIG. 16 presents data on the ability of Vpr to down-regulate the lymphoproliferation of human peripheral blood mononuclear cells (PBMCs) in response to the mitogenic substances: tetanus toxoid, phytohemagglutinin (PHA), concanavalin-A (ConA), and the superantigen staphylococcal enterotoxin B (SEB).

### DESCRIPTION OF PREFERRED EMBODIMENTS OF THE INVENTION

As used herein, the terms “protein” and “polypeptide” are used interchangeably and are intended to refer to proteinaceous compounds including proteins, polypeptides and peptides.

As used herein, the term “individual” refers to the vertebrate targeted for use of the present invention. Examples of “individuals” contemplated by the present invention include but are not limited to humans, higher order primates, canines, felines, bovines, equines, ovines, porcines, avians, and other mammals.

As used herein, the term “immunogenic vector” relates to a vector which elicits an immune response. Examples of immunogenic vectors include, but are not limited to, viral and bacterial vectors. Some embodiments of the present invention relate to methods where the vector administered to the individual is viral. Examples of viral vectors include, but are not limited to, adenoviral vectors; adeno-associated viral vectors; vaccinia viral vectors; SV40 viral vectors; Epstein-Barr virus (EBV) replicon-based vectors (Tsujie et al., 2000, Kidney Int., 59:1390-1396); lentiviral vectors (Buchschacher & Wong-Staal, 2000, Blood, 95:2499-2504; Vigna & Naldini, 2000, J. Gene Med., 2:308-316; Lever, 2000, Curr. Opin. Mol. Ther., 2:488-496) including, but not limited to, HIV-based vectors (Buchschacher & Wong-Staal, 2000, supra) and visna virus vectors (Berkowitz et al., 2001, Virology, 279:116-129); alphavirus vectors (Wahlfors et al., 2000, Gene Ther., 7:472-480), including, but not limited to, Sindbis and Semliki forest virus-based vectors (Perri et al., 2000, J. Virol., 74:9802-9807); and flavivirus vectors (Varnavski & Khromykh, 1999, Virology, 255:366-375). Each of the aforementioned viral vector references is incorporated herein in its entirety by reference. In a preferred embodiment, the vector is adenovirus. Some embodiments of the present invention relate to methods where the vector administered to the individual is bacterial. Examples of bacterial vectors include, but are not limited to, Salmonella, mycobacterium and BC. Examples of immunogenic vectors which are useful in gene therapy and which can be adapted to the present invention include recombinant adenoviral vectors which are described in U.S. Pat. No. 5,756,283 and U.S. Pat. No. 5,707,618, which are each incorporated herein by reference.

As used herein term “desired polypeptide” refers to the polypeptide for which gene therapy is desired Examples of “desired polypeptides” include human and non-human polypeptides. Examples of human polypeptides contemplated by the present invention include, but are not limited to, insulin, growth hormone, the cystic fibrosis polypeptide.

As used herein, the terms “administration” and “administering” refer to the delivery of polypeptides to an individual. “Administration” and administering refer to the delivery of nucleic acids which encode polypeptides and also to the delivery of polypeptides to the individual. The terms include, but are not limited to, delivery routes including intramuscularly, intravenously, intranasally, intraperatoneally, intradermally, intrathecally, intraventricularly, subcutaneously, transdermally, topically, or by lavage. Modes of administration contemplated by this invention include, but are not limited to, the use of a syringe, intravenous line, transdermal patch, or needleless injector.

The present invention provides improved gene therapy vectors that employ one of the weapons that the HIV virus uses to evade and undermine an infected individual's immune system: the Vpr protein and/or a nucleic acid molecule that encodes it. Armed with this HIV-derived weapon, gene therapy vectors can be made more effective by reducing an individual's immune response again them. Moreover, the present invention uses the HIV Vpr protein, and/or a nucleic acid molecule that encodes it, to treat individuals who have diseases and conditions associated with undesirable immune responses.

The present invention arises from the surprising discovery that the delivery of Vpr polypeptide suppresses cellular immune responses. Vpr suppresses CC chemokines and compromises CD8⁺ T cell effector function. This has been shown in both mouse and human systems. Moreover, Vpr inhibits the synthesis of prototypic Th1 type cytokines and shifts the antibody response toward a Th2 type bias. The data support the conclusion that Vpr interferes with costimulatory molecules involved in immune activation. This has been shown in both mouse and human systems. It has also been discovered that Vpr can suppress lymphoproliferation in response to a variety of mitogenic substances including superantigens. Accordingly, when delivered in the context of a gene therapy protocol Vpr decreases the immune response directed at the gene therapy vector, and cells infected by the same, resulting in an increase in the efficacy of the gene therapy protocol. When delivered to an individual who has a disease or condition associated with an undesirable immune response such as an inflammatory or autoimmune disease or tissue or organ transplant, Vpr decreases the immune response.

Infection with adeno-associated virus results in the growth arrest and cell death of newly-established cultures of malignant human tumors, and to have transient antiproliferative effect on diploid human fibroblasts (Bantel-Schaal & Stohr, 1992, J. Virol., 66:773-779, which is incorporated herein by reference). It has now been discovered tat recombinant adenovirus infection induces cell cycle arrest in tumor-derived cells, and that expression of Vpr enhances this effect, to induce a dramatic accumulation of cells in the G2/M phase of the cell cycle. Accordingly, the delivery of Vpr in the context of viral vectors or recombinant viral particles having cell growth arresting properties, can be used to enhance cell cycle arrest for the treatment of diseases characterize by hyperproliferating cells, including, but not limited to cancer.

The amino acid sequence of Vpr and the DNA sequence that encodes it are described in U.S. Pat. No. 5,874,225, issued on Feb. 23, 1999, which is incorporated herein by reference, including the patents and publications referred to therein. Functional fragments of Vpr are described in PCT/US94/02191, filed Feb. 22, 1994, PCT/US95/12344, filed Sep. 21, 1995, and PCT/US98/16890, filed Aug. 14, 1998, which are each incorporated herein by reference, together with the respective corresponding U.S. National Stage applications claiming priority thereto, and U.S. Pat. No. 5,763,190, issued Jun. 9, 1998, which is incorporated herein by reference. U.S. Ser. No. 08/167,608, filed Dec. 15, 1993, and PCT/US94/00899, filed Jan. 26, 1994, which are each incorporated herein by reference, describe recombinant viral particles which include functional fragments of Vpr protein as part of the viral particle.

One aspect of the present invention relates to methods of delivering a desired polypeptide to an individual comprising administering to the individual an immunogenic vector comprising a nucleic acid encoding the desired polypeptide operably linked to regulatory elements in combination with either the Vpr polypeptide, or a functional fragment thereof, or a nucleic acid encoding Vpr, or a functional fragment thereof, operably linked to regulatory elements, or a combination thereof. According to one aspect of the invention, Vpr protein, or a functional fragment thereof, is delivered to an individual in combination with the delivery of an immunogenic vector for delivering the coding sequence of a desired protein in a gene therapy protocol. The Vpr may be delivered as a protein, or a functional fragment thereof, or as a nucleic acid molecule with the coding sequence for Vpr protein, or a functional fragment thereof, or any combination thereof. The Vpr may be delivered in the same formulation as the gene therapy vector or separately. The Vpr may be delivered simultaneously, prior to or subsequent to delivery of the gene therapy vector. In some preferred embodiments, the immunogenic vector comprises a nucleic acid molecule with the coding sequence for Vpr protein or a functional fragment thereof. In some preferred embodiments, the immunogenic vector comprises Vpr protein or a functional fragment thereof. In some preferred embodiments, the immunogenic vector comprises a nucleic acid molecule with the coding sequence for Vpr protein and/or a functional fragment thereof and Vpr protein and/or a functional fragment thereof itself. Once delivered to the individual, the nucleic acid encoding the desired polypeptide is expressed and the desired polypeptide is synthesized within the individual. The presence of the Vpr protein, either delivered as a protein or as a nucleic acid molecule “prodrug” which is expressed, inhibits the immune response directed at the immunogenic vector.

The preset invention provides improved gene therapy compositions and methods. Through gene therapy, polypeptides which are either absent, produced in diminished quantities, or produced in a mutant form in an individual may be replaced using a vector comprising a nucleic acid encoding the desired polypeptide. The desired polypeptide compensates for the lack of the desired polypeptide. Upon administration of the vector to the individual, the individual generates an immune response against the vector. The delivery of Vpr, either a protein or as a nucleic acid molecule “prodrug”, in combination with the gene therapy vector that encodes the desired polypeptide inhibits the immune response directed at the immunogenic vector and therefore increases the efficacy of the gene therapy treatment.

The present invention also provides a method of treating individuals suffering from diseases and conditions characterized by undesirable immune responses such as autoimmune/inflammatory diseases and condition and organ/tissue/cell transplantation procedures. According to the invention, methods of treating an individual with a disease or condition associated with an undesirable immune response comprise administering to the individual Vpr protein or a functional fragment thereof or a nucleic acid encoding Vpr protein or a functional fragment thereof or a combination of two or more of the same. When a nucleic acid encoding Vpr protein or a functional fragment thereof is delivered to an individual, the coding sequence is operably linked to regulatory elements. The Vpr may be delivered as a protein or a functional fragment thereof or as a nucleic acid molecule with the coding sequence for Vpr protein or a functional fragment thereof any combination thereof. In some embodiments, the Vpr is delivered as a nucleic acid molecule with the coding sequence for Vpr protein and/or a functional fragment thereof. In some embodiments, the Vpr and/or a functional fragment thereof is delivered as a protein. Once delivered to the individual, the presence of the Vpr protein, either delivered as a protein or produced by the expression of the nucleic acid molecule tat encodes it, inhibits the undesirable immune response.

As used herein, “inhibit” in reference to an undesirable immune response, refers to any interference with the undesirable immune response resulting in a decrease of the response. For example, the term “inhibit” in this context includes both the elimination and reduction of the undesirable immune response.

According to some embodiments of the present invention, methods are provided for treating individuals suffering from autoimmune diseases and disorders. T cell mediated autoimmune diseases include rheumatoid arthritis (RA), multiple sclerosis (MS), Sjogren's syndrome, sarcoidosis, insulin dependent diabetes mellitus (IDDM), autoimmune thyroiditis, reactive arthritis, ankylosing spondylitis, scleroderma, polymyositis, dermatomyositis, psoriasis, vasculitis, Wegener's granulomatosis, Crohn's disease and ulcerative colitis. Each of these diseases is characterized by T cell receptors that bind to endogenous antigens and initiate the inflammatory cascade associated with autoimmune diseases.

According to some embodiments of the present invention, methods are provided for treating individuals who require immunosuppression such as those undergoing transplantation procedure including cell, tissue and organ transplants. In such instances rejection of the transplanted material is reduced and the severity or incidence of side effects such as graft versus host disease may be lessened.

According to other embodiments, immune suppression can be induced to prevent damage resulting from inflammation. For example, following spinal cord injuries, a cascade of events leads to inflammation of the spinal cord and surrounding tissues. Use of the present invention may both inhibit inflammation of the spinal cord and associated problems and allow delivery of a therapeutic polypeptide (give example) to the individual. For instance, if an axonal guidance protein is the desired polypeptide, use of the present invention may both inhibit the inflammation of the spinal cord and stimulate axonal regrowth.

According to further embodiments of the invention, methods are provided for inhibiting an undesirable immune response in an individual wherein the inhibition effectuates a prophylactic and/or therapeutic treatment of the individual against diseases in which toxins induce the overproduction of inflammatory cytokines such as IL-1 and TNFα. Such diseases include, but are not limited to, septic shock or sepsis, in particular bacterial septic shock or sepsis, and toxic shock, in particular bacterial toxic shock.

As used herein, “septic shock” refers to the systemic symptoms that occur in an individual due to the high levels of inflammatory cytokines secreted principally by macrophages in response to endotoxin exposure, including, but not limited to exposure to gram-negative endotoxin, also known as lipopolysaccharide (LPS). These systemic symptoms include decreased blood pressure, fever, diarrhea, hypoglycemia, and widespread blood clotting.

As used herein, “toxic shock” refers to the systemic symptoms that are quite similar to septic shock, but are triggered by superantigen-induced activation and expansion of T cells. Toxic shock includes, but is not limited to, the food poisoning effects of Staphylococcus aureus exotoxins SEA, SEB, SEC, SED, and SEE, as well as, the toxic shock syndrome caused by Staphylococcus aureus TSST1, often associated with wound infection and tampon use.

As used herein, the term “prophylactic” in reference to treatment of an undesirable immune response, means that the component is administered prior to an undesirable immune response, and that the treatment prevents the occurrence of the undesirable immune response, or decreases the magnitude of the undesirable immune response if it does occur. Preferably, the prophylactic treatment prevents mortality due to the undesirable immune response.

As used herein, the term “therapeutic” in reference to treatment of an undesirable immune response, means that the component is administered during the undesirable immune response, and that the treatment relieves or lessens that undesirable immune response. Preferably, the prophylactic treatment prevents mortality due to the undesirable immune response.

According to other embodiments of the invention, methods are provided for arresting the growth of cells (inhibiting cellular proliferation), such as hyperproliferating cells, such as cancer.

As used herein, the term “inhibit” in reference to the cellular proliferation of a cell, refers to disruption of a cell's progression through the cell cycle. Inhibition of cellular proliferation can be monitored by such means as cell cycle analysis by fluorescence activated cell sorting of propidium iodide-stained cells, or by use of animal tumor models, wherein the size, rate of growth or other characteristics of tumors may be assessed.

As used herein, “anti-tumor fragment” in reference to Vpr protein, refers to fragments of Vpr that will inhibit cellular proliferation of a replicating cell.

As discussed above, in some embodiments, Vpr is delivered alone and in some embodiments, Vpr is delivered in combination with a therapeutic gene including immunogenic vectors.

In some embodiments of the present invention, a combination of one or more of Vpr, a functional fragment thereof, a nucleic acid encoding Vpr, or a nucleic acid encoding a functional fragment of Vpr is administered to a patient.

In some embodiments of the present invention, Vpr or a functional fragment thereof is administer as a protein. In some embodiments, the Vpr or a functional fragment thereof is administered to the individual in the same formulation as the nucleic acid encoding the desired polypeptide. In other embodiments, the Vpr or a functional fragment thereof is administered to the individual in a separate formulation than the nucleic acid encoding the desired polypeptide. In some embodiments, the formulation containing the Vpr or a functional fragment thereof is administered to the individual at the same time as the formulation containing the nucleic acid encoding the desired polypeptide. In some embodiments, Vpr or a functional fragment thereof is delivered as a protein incorporated within an immunogenic vector.

In some embodiments of the present invention, a nucleic acid that encodes Vpr or a functional fragment thereof is administered. In some embodiments of the present invention, the desired polypeptide is encoded by a first nucleic acid while the Vpr or a functional fragment thereof is encoded by a second nucleic acid. In some embodiments, the nucleic add that encodes Vpr or a functional fragment thereof is administered to the individual in the same formulation as the nucleic acid encoding the desired polypeptide. In other embodiments, the nucleic acid that encodes Vpr or a functional fragment thereof is administered to the individual in a separate formulation than the nucleic acid encoding the desired polypeptide. In some embodiments, the formulation containing the nucleic acid that encodes Vpr or a functional fragment thereof is administered to the individual at the same time as the formulation containing the nucleic acid encoding the desired polypeptide. In a preferred embodiment of the present invention, the nucleic acid that encodes Vpr or a functional fragment thereof and the desired polypeptide are encoded by the same nucleic acid which is a genome of an immunogenic vector. In some embodiments, the nucleic acid that encodes Vpr or a functional fragment thereof is administered free of an immunogenic vector that encodes a desired polypeptide.

In some embodiments, the Vpr coding sequence is delivered separately from or free of an immunogenic vector. Compositions and methods for delivering proteins to cells by direct DNA administration have been reported using a variety of protocols. Examples of such methods are described in U.S. Pat. No. 5,593,972, U.S. Past No. 5,739,118, U.S. Pat. No. 5,580,859, U.S. Pat. No. 5,589,466, U.S. Pat. No. 5,703,055, U.S. Pat. No. 5,622,712, U.S. Pat. No. 5,459,127, U.S. Pat. No. 5,676,954, U.S. Pat. No. 5,614,503, and PCT Application PCT/US95/12502, which are each incorporated herein by reference. Compositions and methods for delivering proteins to cells by direct DNA administration are also described in PCT/US90/01515, PCT/US93/02338, PCT/US93/048131, and PCT/US94/00899, which are each incorporated herein by reference. In addition to the delivery protocols described in those applications, alternative methods of delivering DNA are described in U.S. Pat. Nos. 4,945,050 and 5,036,006, which are both incorporated herein by reference. Nucleic acid molecules can also be delivered using liposome-mediated DNA transfer such as that which is described in U.S. Pat. No. 4,235,871, U.S. Pat. No. 4,241,046 and U.S. Pat. No. 4,394,448, which are each incorporated herein by reference.

Formulations comprising an immunogenic vector comprising the nucleic acid having a sequence encoding the desired polypeptide are made up according to the mode and site of administration. Such formulation is well within the skill in the art. In addition to nucleic acids and optionally polypeptides, the formulation may also include buffers, excipients, stabilizers, carriers and diluents.

The pharmaceutical composition comprising Vpr protein or a fragment thereof and a pharmaceutically acceptable carrier or diluent may be formulated by one having ordinary skill in the art with compositions selected depending upon the chosen mode of administration Suitable pharmaceutical carriers are described in the most recent edition of Remington's Pharmaceutical Sciences, A. Osol, a standard reference text in this field, which is incorporated herein by reference.

For parenteral administration, the Vpr protein can be, for example, formulated as a solution, suspension, emulsion or lyophilized powder in association with a pharmaceutically acceptable parenteral vehicle. Examples of such vehicles are water, saline, Ringer's solution, dextrose solution, and 5% human serum albumin Liposomes and nonaqueous vehicles such as fixed oils may also be used The vehicle or lyophilize powder may contain additives that maintain isotonicity (e.g., sodium chloride, mannitol) and chemical stability (e.g., buffers and preservatives). The formulation is sterilized by commonly used techniques. For example, a parenteral composition suitable for administration by injection is prepared by dissolving 1.5% by weight of active ingredient in 0.9% sodium chloride solution.

The pharmaceutical compositions comprising Vpr protein, or fragments thereof may be administered by any means that enables the active agent to reach the agent's site of action in the body of a mammal. Because proteins are subject to being digested when administered orally, parental administration, i.e., intravenous, subcutaneous, intramuscular, would ordinarily be used to opt absorption.

The dosage administered varies depending upon actors such as: pharmacodynamic characteristics; its mode and route of administration; age, health, and weight of the recipient; nature and extent of symptoms; kind of concurrent treatment; and frequency of treatment. Usually, a daily dosage of Vpr protein can be about 0.1 to 100 milligrams per kilogram of body weight. Ordinarily 0.5 to 50, and preferably 1 to 10 milligrams per kilogram per day given in divided doses 1 to 6 times a day or in sustained release form is effective to obtain desired results.

Another aspect of the present invention relates to pharmaceutical compositions that comprise a nucleic acid molecule that encodes Vpr and a pharmaceutically acceptable carrier or diluent. According to the present invention, genetic material that encodes Vpr protein is delivered to an individual in an expressible form. The genetic material, DNA or RNA, is taken up by the cells of the individual and expressed. Vpr that is thereby produced can inhibit immune responses, either those directed at an immunogenic vector or another undesirable immune response such as those associated with autoimmune and inflammatory disease and conditions and transplantation procedures. Thus, pharmaceutical compositions comprising genetic material that encodes Vpr are useful in the same manner as pharmaceutical compositions comprising Vpr protein. Vpr or nucleic acid molecule with a Vpr coding sequence may be incorporated into an immunogenic vector.

Nucleotide sequences that encode Vpr protein operably linked to regulatory elements necessary for expression in the individual's cell may be delivered as pharmaceutical compositions using a number of strategies which include, but are not limited to, either viral vectors such as adenovirus or retrovirus vectors or direct nucleic acid transfer. Methods of delivery of nucleic acids encoding proteins of interest using viral vectors are widely reported. A recombinant viral vector such as a retrovirus vector or adenovirus vector is prepared using routine methods and staring materials. The recombinant viral vector comprises a nucleotide sequence that encodes Vpr. Such a vector is combined with a pharmaceutically acceptable carrier or diluent. The resulting pharmaceutical preparation maybe administered to an individual. Once an individual is infected with the viral vector, Vpr is produced in the infected cells.

Alternatively, a molecule which comprises a nucleotide sequence that encodes Vpr can be a as a pharmaceutical composition without the use of infectious vectors. The nucleic acid molecule may be DNA or RNA, preferably DNA. The DNA molecule may be linear or circular, it is preferably a plasmid. The nucleic acid molecule is combined with a pharmaceutically acceptable carrier or diluent.

According to the invention, the pharmaceutical composition comprising a nucleic acid sequence that encodes Vpr protein may be administered directly into the individual or delivered ex vivo into removed cells of the individual which are reimplanted after administration. By either route, the genetic material is introduced into cells which are present in the body of the individual. Preferred routes of administration include intramuscular, intraperitoneal, intradermal and subcutaneous injection. Alternatively, the pharmaceutical composition may be introduced by various means into cells that are removed from the individual. Such means include, for example, transfection, electroporation and microprojectile bombardment. After the nucleic acid molecule is taken up by the cells, they are reimplanted into the individual.

The pharmaceutical compositions according to this aspect of the present invention comprise about 1 ng to 10 mg of nucleic acid in the formulation; in some embodiments, about 0.1 to about 2000 micrograms of DNA. In some preferred embodiments, the pharmaceutical compositions contain about 1 to about 1000 micrograms of DNA. In some preferred embodiments, the pharmaceutical compositions contain about 1 to about 500 micrograms of DNA. In some preferred embodiments, the pharmaceutical compositions contain about 25 to about 250 micrograms of DNA. Most preferably, the pharmaceutical compositions contain about 100 micrograms DNA.

The pharmaceutical compositions according to this aspect of the present invention are formulated according to the mode of administration to be used. One having ordinary skill in the art can readily formulate a nucleic acid molecule that encodes Vpr. In cases where injection is the chosen mode of administration, a sterile, isotonic, non-pyrogenic formulation is used. Generally, additives for isotonicity can include sodium chloride, dextrose, mannitol, sorbitol and lactose. Isotonic solutions such as phosphate buffered saline are preferred. Stabilizers include gelatin and albumin.

Regulatory elements for nucleic acid expression include promoters, initiation codons, stop codons, and polyadenylation signals. It is necessary hat these regulatory elements be operably linked to the sequence that encodes the desired polypeptides and optionally the Vpr polypeptide and that the regulatory elements are operable in the individual to whom the nucleic acids are administered. For example, the initiation and termination codons must be in frame with the coding sequence. Promoters and polyadenylation signals used must also be fictional within the cells of the individual.

Examples of promoters useful to practice the present invention include, but are not limited to, promoters from Simian Virus 40 (SV40), Mouse Mammary Tumor Virus V Human Immunodeficiency Virus (HIV) such as the HIV Long Terminal Repeat (LTR) promoter, Moloney murine leukemia virus (M-MuLV), avian leukosis virus (ALV), Cytomegalovirus (CMV) such as the CMV immediate early promoter, Epstein Barr Virus (EBV), Rous Sarcoma Virus (RSV), as well as promoters from human genes such as human actin, human myosin, human hemoglobin, human muscle creatine kinase (MCK), and human metallothionein.

Examples of polyadenylation signals useful to practice the present invention include, but are not limited to, SV40 polyadenylation signals and retroviral LTR polyadenylation signals. In particular, the SV40 polyadenylation signal which is in pCEP4 plasmid (Invitrogen, San Diego Calif.), referred to as the SV40 polyadenylation signal, is used.

### EXAMPLES

### Example 1

### HIV-1 Vpr Suppresses CC Chemokines and Compromises CD8⁺ Effector Function In Vivo

### Methods

Effect of Vpr on cell surface molecules assessed by flow cytometry: Recombinant Vpr protein containing supernatant and control supernatants were prepared and the concentration of Vpr in the supernatant was determined by Levy et al., 1995, J. Virol., 69:1243-1252, which is incorporated herein by reference. Normal human peripheral blood mononuclear cells (PBMC) were purified by standard Ficoll-Hypaque and the lymphocytes were stimulated with 5 μg/mL of phytohemagglutinin (PHA) and then treated with different concentrations of Vpr protein. Twenty-four and 48 hours post treatment, 2×10⁵ cells were stained with fluorescein isothiocyanate-(FITC) or phycoerythrin-(PE) labeled monoclonal antibodies (mAbs) to human CD3, CD4, CD8, CD25, CD28, CD40, CD45RA, CD80, CD86, HLA-DR, and CTLA-4 (Pharngen, San Diego, Calif.), followed by washes in phosphate-buffered saline (PBS). Cells were fixed with 2% paraformaldehyde and analyzed by a fluorescence activated cell sorter (FACS) (Becton-Dickinson, CA).

Effect of Vpr on β-chemokine production and chemokine receptor CCR-5: Normal human PMBCs were stimulated with 5 μg/mL of PHA and then treated with different concentrations of Vpr protein Twenty-four and 48 hours post treatment, supernatants were collected and assayed for β-chemokine production by ELISA using a combination of capture and demon antibodies. Recombinant proteins (standards) and capture and detection antibodies were purchased from Intergen (Purchase, N.Y.), and the assay was performed according to the manufacturer's instructions. Vpr-treated cells were analyzed for CCR-5 expression by double-staining with anti-Mac and anti-CCR-5 mAbs purchased from Pharmingen (San Diego, Calif.). Cells were analyzed by FACS and the data were peed using Cell Quest Software (Becton Dickinson, CA).

### Results

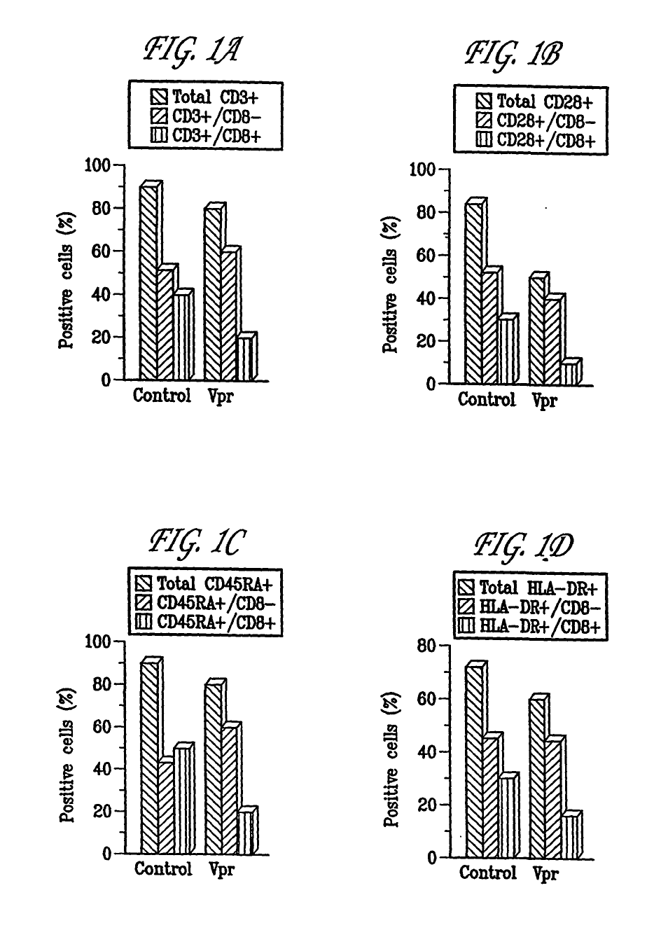

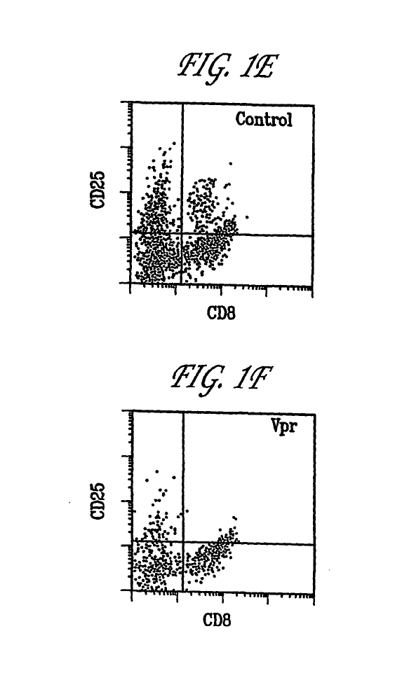

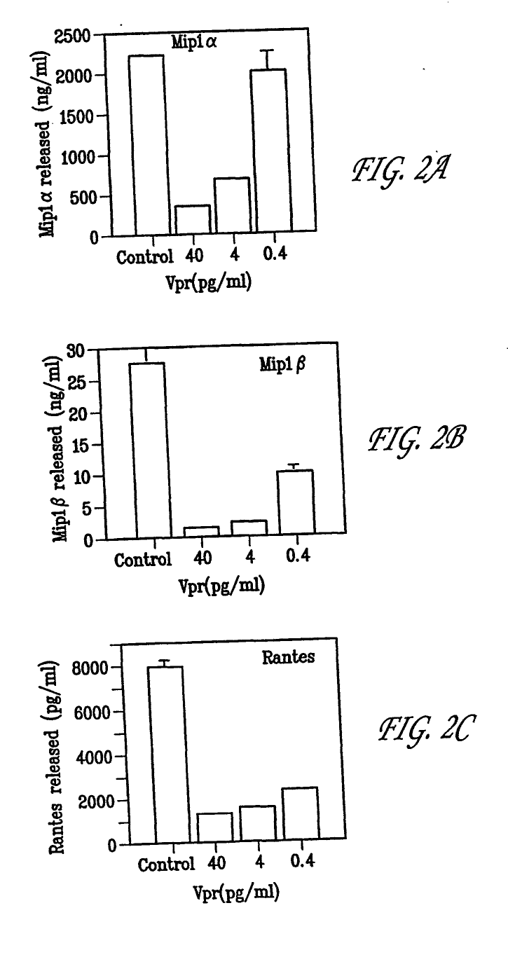

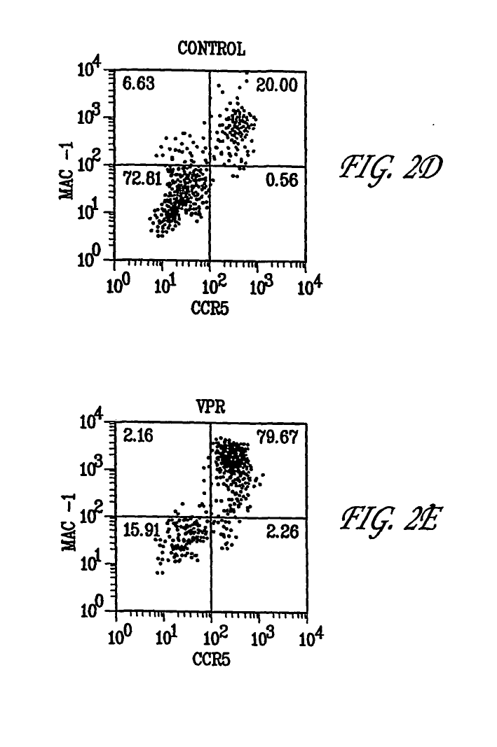

FIGS. 1A, 1B, 1C, 1D, 1E and 1F show the effect of treatment with Vpr on cell subsets. FIGS. 1A, 1B, 1C and 1D show a 50% reduction in the number of CD8⁺ T cells expressing CD28, CD45RA, and HLA-DR following treatment with 40 pg/mL Vpr. FIGS. 1E and 1F show a significant reduction in the number of CD8⁺ T cells expressing activation maker CD25 (IL-2R) following treatment of activated peripheral blood mononuclear cells (PBMCs) with Vpr.

FIGS. 2A, 2B, 2C, 2D and 2E show the effect of element with Vpr on PBMCs. FIGS. 2A, 2B and 2C show that treatment with Vpr decreases the secretion of MIP-1α, MIP-1β, and RANTES. FIGS. 2D and 2E shows ta Vpr treatment of PBMCs increased the expression of the chemokine receptor CCR-5 significantly.

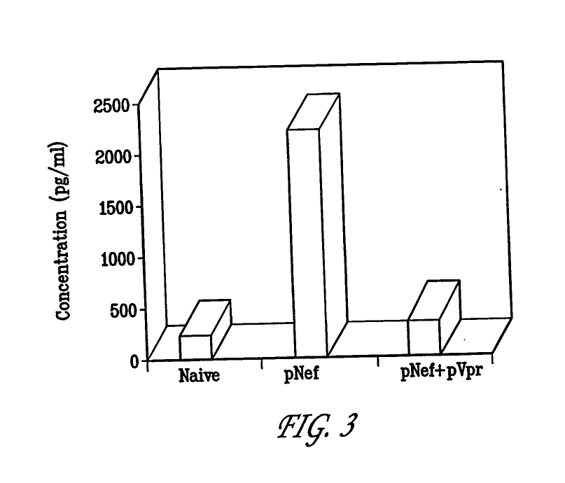

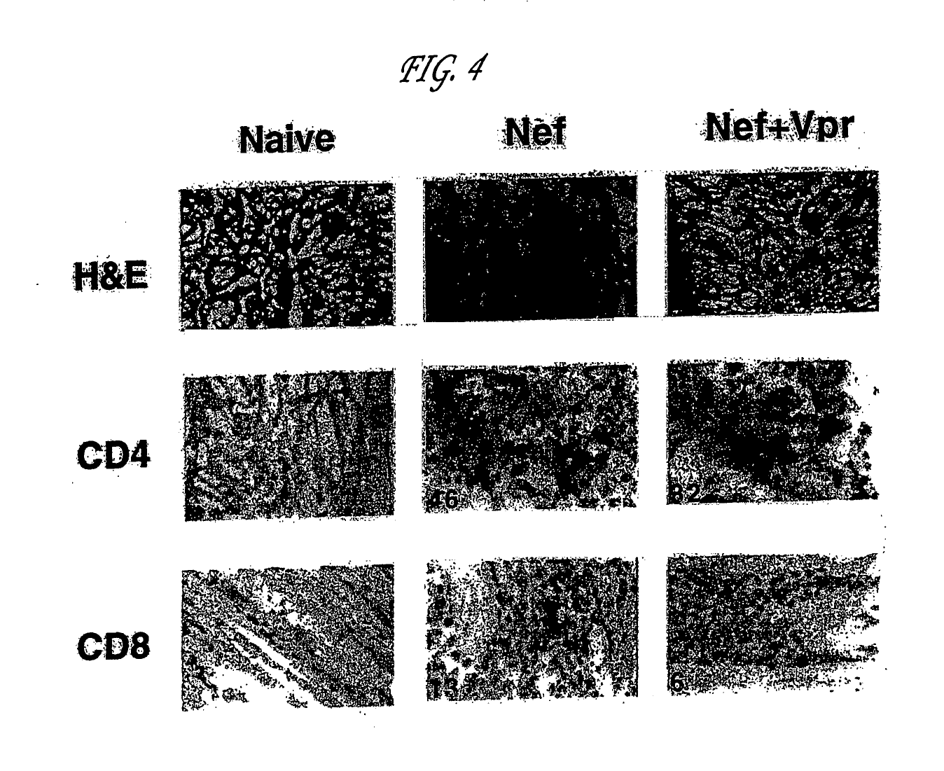

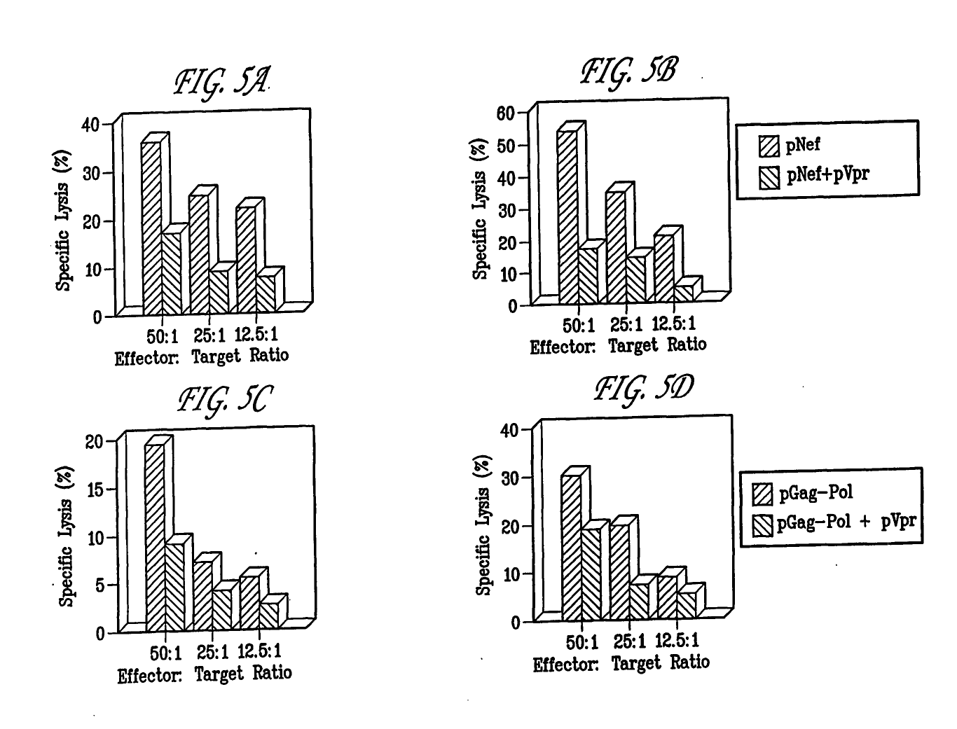

FIG. 3 depicts MIP-1α production in splenocytes from mice immunized with pNef with or without co-immunization of pVpr. Splenocytes from mice coimmunized with pNef and pVpr produced significantly less MIP-1α than did splenocytes from mice immunized with pNef and control vector.

### Example 2

### Vpr Specifically Interferes with the Induction of Cell-Mediated Immune Responses In Vivo

To investigate the effect of Vpr on immune activation in vivo, a DNA vaccine model system was used. DNA immunization has been used to induce immune responses to foreign antigens of interest in vivo through inoculation of the host with plasmids encoding pathogens or tumor antigens (Tang et al., 1992, Nature, 356:152-4; Ayyavoo et al., 1997, AIDS, 11:1433-44; Kim et al., 1998, Oncogene, 17:3125-35, each of which is incorporated herein by reference). In vivo injection of plasmid results in protein production in local transfected muscle cells as well as in directly transfected APCs (Kim et al., 1998, supra; Chattergoon et al., 1998, J. Immunol., 160: 5707-5718; Manickan, 1997. J. Leukoc. Biol., 61:125-132; Condon, 1996, Nat. Med., 2: 1122-1128, each of which is incorporated herein by reference). This technique elicits both humoral and cellular responses to the specific immunizing antigens in animal models and humans (Letvin et al., 1997, Proc. Natl. Acad. Sci. USA, 94: 9378-9383; Torres, et al., 1999, Vaccine, 18: 805-814; Boyer et al., 1999, Clin. Immunol., 90: 100-107, each of which is incorporated herein by reference). To investigate Vpr modulation of immune activation in vivo, mice were co-immunized with different HIV-1 plasmid encoded antigens in the presence and absence of Vpr plasmid and measured the immune responses (cellular and humoral) induced by the immunizing antigen. The results demonstrate that Vpr specifically interferes with the induction of cell-mediated immune responses in vivo. Furthermore, Vpr specifically inhibited the synthesis of prototypic Th1 type cytokines and shifted the antibody response towards a Th2 type bias. Further studies demonstrated that Vpr either as recombinant protein or as virion-associated molecule down regulated the expression of CD40 and CD80, but not CD11a supporting that Vpr specifically interferes with costimulatory molecules involved in immune activation. These data support that in vivo Vpr can specifically and significantly interfere with the development of antigen specific immunity.

### Materials and Methods

Cells: HeLa, RD, and NIH3T3 cells, obtained from the American Type Culture Collection (ATCC, Manassas, Va.), were grown in monolayers, at 37° C. in 5% CO₂, in Dulbecco's modified Eagle's medium (DMEM), 10% fetal bovine serum (FBS), 1% penicillin, 1% streptomycin and 1% L-glutamine. P815 cells, obtained from ATCC, were maintained as suspension cultures in RPMI 1640, 10% FBS, 1% penicillin, 1% streptomycin and 1% L-glutamine, at 37° C. with 5% CO₂.

Macr phage preparation: BALB/c (female, 8-week old) mice were injected with RPMI 1640 and the peritoneal macrophages were isolated by lavage method (Shirtsuchi et al., 1998, Biochem. Biophys. Res. Commun., 246:549-555, which is incorporated herein by reference). The cells in the fluid were collected by centrifugation, washed with Hank's balanced salt solution (Life Technologies Inc. Rockville, Md.), and cultivated in a 6 well plate with RPMI1640 medium, containing 10 mM Hepes (pH 7.0). Non-adherent cells were washed out of the plate, and the remaining adherent cells were removed from the plate and analyzed for surface antigen expression by FACS analysis.

Cloning and expression of DNA vaccine constructs: Plasmids expressing HIV-1 antigens Vpr, Nef, Gag-pol and Vif were constructed using appropriate polymerase chain reaction (PCR) primers as described (Ayyavoo, et al., 1997, Sa, and Mahalingam et al., 1997, J. Virol., 71:6339-6347, which are incorporated herein by reference). All the plasmids were sequenced to verify the coding region, and further analyzed for protein expression by immunoprecipitation using specific antibodies. To further examine the expression and trafficking of Vpr antigen in vivo, we have fused Vpr in frame with green fluorescent protein (GFP) and cloned vpr-GFP into a eukaryotic expression vector as described (Muthumani et al., 2000, DNA Cell Biol., 19:179-188, which is incorporated herein by reference).

Mice: BALB/c female mice, aged 6-8 weeks, were purchased from Harlan Sprague Dawley, Inc., (Indianapolis, Ind.). The mice were housed in a temperature-controlled, light-cycled room, as per the guidelines of National institutes of Health and the University of Pennsylvania.

DNA inoculation: A facilitated DNA inoculation protocol which results in increased protein expression levels from plasmid-delivered genes in vivo was utilized. Specifically, the quadriceps muscles of BALB/c mice were injected with 100 μl of 0.25% bupivacaine-HCL (Sigma, MO) using a 27-gauge needle. Forty eight hours later, 100 μg of the DNA construct of interest, in PBS, was injected into the same region of the muscle as the bupivacaine injection. Mice were given one injection followed by a boost two weeks later. Two weeks after the second injection, half of the mice in each group were sacrificed for their spleens, and the remaining mice were given a second boost with the appropriate DNA construct.

In vivo expression of Vpr by immunostaining. Eight- to ten-week-old BALB/c mice were immunized with 100 μg of Vpr-GFP expression plasmid (pcVpr-GFP) or control vector (pcDNA3) as described above. Three days post injection mice were sacrificed and the quadriceps muscle and regional lymph node (popliteal & inguinal femoral) were removed. Muscle and lymph nodes were cryopreserved and 0.2 micron sections were prepared for staining. Staining for Vpr was performed on frozen tissue sections by fixing them with methanol at room temperature for 30 minutes, blocked and incubated for 90 minutes with Vpr-specific antiserum (1:100) or pre-immune serum. After washing with PBS, the sections were incubated for 90 minutes with PE-conjugated affinity purified F(ab)′2 fragment of goat anti-rabbit IgG (ICN Biochemicals, CA) diluted at 1:500 in PBS. Slides were washed with PBS, stained with DAPI (0.1% in PBS; Sigma, St Louis, Mo.), washed again, and mounted using a fade-resistant mounting medium (Ted Pella Inc., Redding, Calif.). All incubations were carried out at 37° C. in a humidification chamber. Hematoxylin and eosin (H&E) staining was performed as described (T. C. Sheehem and B. B. Hrapchak, 1980, Theory and Practice of Histotechnology, St Louis, Mo., C. B. Mosby Co., which is incorporated herein by reference).

Cytotoxic T lymphocyte assay: Recombinant vaccinia viruses (vMN462, vVK1, VV:gag, vTFnet vSC8) were obtained from the NIH AIDS Research and Reference Reagent Program and P815 cell line was obtained from ATCC. A five hour ⁵¹Cr release assay was performed using vaccinia-infected targets. The effectors were stimulated for 24 hours with Con A (Sigma, MO) at 2 μg/ml concentration followed by specific stimulation with vaccinia-infected P815 cells, which were fixed with 0.1% glutaraldehyde for 2-3 days. A standard chromium release assay was performed in which the target cells were labeled with 100 μCi/ml Na₂ ⁵¹CrO₄ for 2 hours and incubated with the stimulated effector splenocytes for 6 hours at 37° C. CTL activity was determined at effector:target (E:T) ratios raging from 50:1 to 5:1. Percent specific lysis was determined from the formula:

100×{experimental release−spontaneous release/maximum release−spontaneous release}.

Maximum release was determined by lysis of target cells in 1% Triton X-100 containing medium

ELISA: Fifty microliters of recombinant Nef (Intracel, MA) or purified prostate specific antigen (PSA) protein (Fitzgerald Industries, MA) diluted in 0.1 M carbonate-bicarbonate buffer (pH 9.5) to 2 μg/ml concentration was adsorbed onto microtiter wells overnight at 4° C. as described (Kim et al., 1998, supra). Mouse sera (pre-immune and post-immune) were diluted and incubated for 1 hour at 37° C., then incubated with horseradish peroxidase (HRP)-conjugated goat anti-mouse IgG (Sigma, MO). The plates were washed and developed with 3′3′5′5′ TMB buffer and the plates were read at OD₄₅₀.

Effect of viron-ass ciated Vpr n mouse peritoneal macrophages: To assess the effect of Vpr on APCs, we infected mouse macrophages with amphotropic pseudotype viruses either containing or lacking Vpr. Constructs pNL43 R⁺E⁻, pNL43 R⁺E⁻ and pVSV-G-Env were obtained from NIH AIDS RRRP, which are contributed by Dr. Landau. Virus preparation was done by cotransfecting RD cells with pVSV-G-Env and pNL43.HSA.R⁺E⁻ or pNL43.HSA.R⁺E⁻. Seventy two hours post transfection supernatant was collected, concentrated and assayed for virus production by measuring p24 antigen production. Cells were infected with 10 pg p24 antigen equivalents of viruses (Akkina et at., 1996, J. Virol., 70:2581-2585, which is incorporated herein by reference). VSV-G-Env-complemented pseudotype viruses infect macrophages in single round infection, which allows the study of the virion-associated Vpr-mediated effects. Forty eight hours post infection cells were analyzed for the expression of costimulatory molecules (CD40, CD80) and surface antigens (CD111a) as described below.

Multicolor Flow Cytometry Analysis: Single cell suspensions were washed in PBS (pH 7.2) containing 0.2% bovine serum albumin and 0.1% NaN₃. Cells were incubated with goat IgG to block binding of Ig to FcγR and stained with FITC-, PE- or Cy5-labeled antibodies and IgG control antibody for 60 minutes. Cells were washed with PBS and fixed with 2% paraformaldehyde and analyzed using a fluorescence activated cell sorter (Beckton-Dickinson, CA). FITC-, PE- or Cy5-labeled mouse mAbs to CD11a, CD80 and CD40 were purchased from PharMingen, C A and Coulter, F L. Data were analyzed using CELL Quest program (Beckton-Dickinson, CA).

### Results

Effect of Vpr on Mouse Cells: The effect of Vpr on immune activation in vivo was examined using a mouse model. Although Vpr is known to alter the cell cycle events in human cells of different lineages, it is important to demonstrste that HIV-1 Vpr can exert similar effects in murine cells. Both HeLa (human) and NIH3T3 (mouse) cells were transfected with Vpr expression plasmids and compared for Vpr subcellular localization and its ability to inhibit cell proliferation. HeLa and NIH3T3 cells, maintained in Dulbecco's Modified Eagle's medium (DMEM) containing 10% FBS, were transfected with a CMV Vpr expression plasmid or a control vector plasmid. Localization of Vpr was detected by indirect immunofluorescence as described in the methods. Results of the subcellular localization of Vpr in human and murine cells by indirect immunofluorescence assay indicate a similar Vpr localization pattern in both human and murine cells. HeLa and NIH3T3 cells were transfected with HIV-1-Vpr expression vector, stained with anti-Vpr antibody followed by PE-conjugated goat anti-rabbit secondary antibody. The same fields were stained by DAPI (nuclear) staining. In an effort to further confirm whether Vpr exhibits a similar localization pattern in mouse primary cells, mouse peritoneal macrophages were infected with HIV-1 complemented with VSV-G-Env and stained for Vpr localization. Results of the subcellular localization of virion-associated Vpr mouse peritoneal macrophages indicate that in mouse primary cells Vpr showed a nuclear localization pattern similar to that in human pry cells. Mouse peritoneal macrophages (4×10⁶ cells) were isolated and infected with VSV-G-Env complemented HIV-1 vpr⁺ and HIV-1 vpr⁻ viruses. Infected cells containing virion-associated Vpr were visualize by indirect immunofluorescence using Vpr-specific antibody and photographed under FITC filter.

In addition to the cellular localization of Vpr in human and mouse cells, the effect of Vpr for one of its well-characterized biological functions, cell-cycle arrest was also tested. The ability of different Vpr molecules (which differ in their biological function) to inhibit cell proliferation in human and murine cell lines was tested. The results are shown in Table 1. These results indicate that Vpr molecules modulate cell proliferation in both human and murine cell lines in a similar manner, suggesting that Vpr exerts similar effects on the basic cellular machinery of each cell type. Taken together, the localization and cell proliferation analyses in these two cell lines support the appropriateness of a murine model for further in vivo immune studies.

In vivo expression of Vpr in mouse tissues: Six- to ten-week-old mice were immunized with 50 μg of Vpr-GFP expression plasmid, GFP expression plasmid, or the control vector plasmid in quadricepts muscle. Mice were sacrificed after 72 hours and the quadricepts muscle and the regional lymph node were removed and frozen in OCT (Vector Laboratories Inc., CA). The frozen sections were cut into 0.2 micron sections and expression of Vpr-GFP was visualized directly by fluorescence microscopy under the FITC filter. Results demonstrate that expression of Vpr is detected in both the muscle and in the regional lymph node.

The expression and localization of Vpr in vivo was evaluated. Mice were immunized into the right quadriceps muscles with pVpr-GFP, pGFP or pcDNA3 vector plasmid. Three days post immunization, mice were sacrificed and the right quadriceps muscle was frozen in OTC and sectioned. Sections were viewed directly and photographed for Vpr-GFP expression using FITC filter. Slides from control vector; GFP plasmid; and pVpr-GFP immunized mice muscle sections were compared at 40× magnification. In muscle sections Vpr-GFP showed a dramatic and distinct nuclear staining comparable to GFP staining. As expected, staining with pre-immune sera or staining of sections from the control vector-immunized mice did not show any specific staining. These results indicate that in vivo expression of Vpr does result in nuclear localization in the muscle fiber of a living animal as expected.

The expression and distribution of Vpr in inguinal lymph nodes in vivo was evaluated. Mice were immunized into the right quadriceps muscle with pVpr-GFP. Five days post immunization, mice were sacrificed and the popliteal and inguinal lymph nodes proximal to the site of injection were harvested and sectioned. Sections were viewed directly under FITC filter for Vpr-GFP expression and photographed The same section was counter stained with anti-Vpr antibodies followed by PE-conjugated goat anti-rabbit secondary antibody. To identify the cell types expressing Vpr-GFP, adjacent section was stained with hematoxylin and eosin.

DNA immunization results in migration of transfected local APCs to the regional lymph node to present antigen to T cells. To identify the cells expressing Vpr antigen in the lymphoid organ (lymph node) in vivo further studies were performed. Direct visualization of Vpr-GFP was performed in lymph node sections obtained from pVpr-GFP inoculated mice. Furthermore, to confirm the specificity of stained cells, we also performed immunofluorescence using specific anti-Vpr antibody or pre-immune sera followed by PE-conjugated anti-rabbit IgG. Further analysis of antigen expressing cell types by H&E staining from these lymph node sections indicates that most of the cells expressing Vpr antigen are ACPs.

Effect of Vpr on lymphocyte recruitment and immune activation in vivo: Injection of antigen expression cats can result in lymphocyte infiltration at the site of injection. Ten-week-old BALB/c mice were co-immunized with a Vpr expression plasmid (pVpr) and different HIV-1 antigen immunization constructs (pGag or pNef). As an example of responses observed with these antigens, pNef immunization in muscle resulted in significant infiltration of lymphocytes and macrophages at the immunization site.

The results shown in FIG. 4 are of immunohistochemical analysis of lymphocyte infiltration at the site of antigen expression. Frozen muscle sections from naïve, pNef; and pNef plus pVpr immunized mice (n=4) were prepared seven days post immunization and stained with anti-CD4 (CD4) and anti CD8 (CD8) antibodies. Row H&E represents the sections stained with hematoxylin and eosin stain for visualization of infiltrating lymphocytes. The nuclei are shown in blue and the cytoplasm is shown in red stain; CD4 and CD8 indicate the sections stained with anti-CD4 or anti-CD8 antibodies. The positive cells are stained brown and the number of positive cells is indicated inside each panel. Five panels were examined for each staining for each experiment. Similar results were obtained in multiple experiments. In order to determine whether Vpr interferes with the trafficking and recruitment of lymphocytes to the inflammatory site, we analyzed muscle sections of mice immunized with pNef in the presence or absence of pVpr on day 7 as described in methods. FIG. 4, row H&E shows that the number of infiltrating lymphocytes is much higher in mice immune with pNef alone or pNef and control plasmid, whereas, pVpr co-immunization reduced lymphocyte infiltration dramatically. To further delineate the effect of pVpr on CD4⁺ versus CD8⁺ cells, specific immunohistochemical staining was performed as shown for CD4 and CD8 cells. The CD4:CD8 ratio was determined by averaging 5 different fields (20× magnification). The CD4:CD8 ratio is lower in pNef immunized mice (2:1) compared to pVpr co-immunization (3:1). Similar results were observed with pGag and pVpr co-immunization also. Overall, it is clearly evident that both T cell populations were affected by pVpr co-immunization.

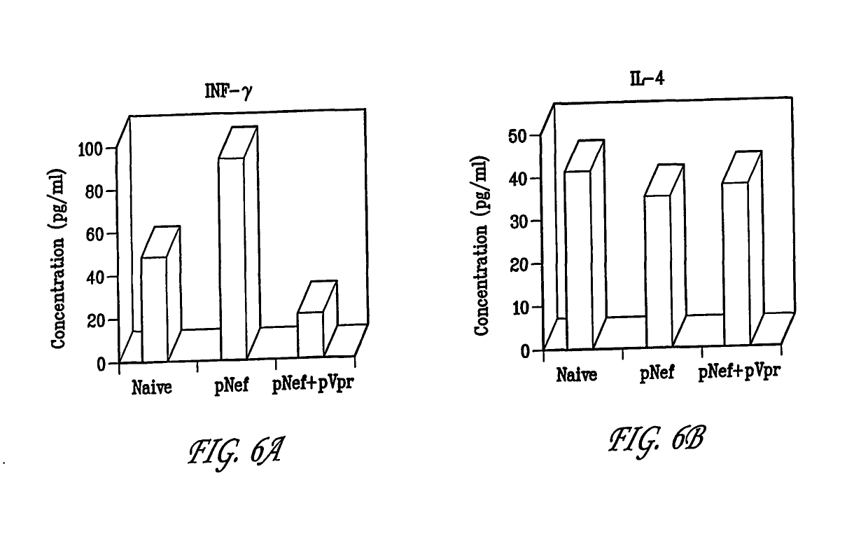

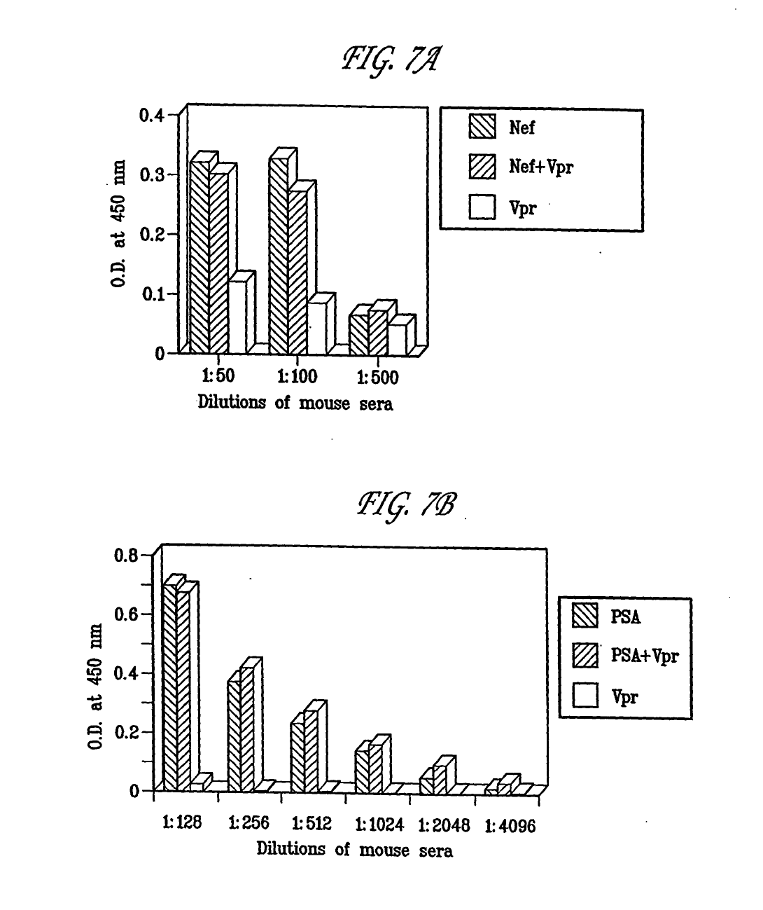

Vpr modulates the antigen-driven CD8⁺ mediated cytotoxic T lymphocyte (CTL) response: To investigate whether a correlation exists between the Vpr-mediated effects on T cell recruitment and cellular immunity, splenocytes collected from mice co-immuned with HIV-1 antigen plasmids and Vpr plasmid were assayed for antigen specific CTL activity. Nef-specific CTL activity measured in pNef and pVpr co-immunized mice was suppressed significantly in comparison to mice immunized with pNef alone. The results are shown in FIGS. 5A, 5B, 5C and 5D which show cytotoxic T lymphocyte response induced by pNef or pGag-Pol in the presence or absence of pVpr co-immunization. BALB/c mice were immunized with 100 μg of pNef and control vector or 100 μg of pNef and pVpr. Splenocyctes were obtained from the mice (n==4) 2 weeks after the first and second boost and antigen specific CTL assay was performed in a 6 hour ⁵¹Cr release method. The graphs represent the percentage of specific lysis induced by subtracting the non specific lysis measured by assay using the target cells infected with control recombinant vaccinia. FIGS. 5A and 5B represent the specific lysis (%) induced by pNef vaccine in the presence or absence of pVpr co-immunization, two weeks after first and second boost, restively. FIGS. 5C and 5D are the same described above except here the antigen expression plasmid is pGag-Pol instead of pNef. These studies were repeated 4 times with similar results. Mice immunized with pNef and control vector exhibited 37% and 53% specific lysis at a 50:1 effector: target ratio after the second and third injections, respectively. In contrast, mice receiving equal amounts of pNef and pVpr resulted in less than 17% and 19% specific lysis at a 50:1 effector: target ratio after second and third immunizations, respectively. Similar results were observed after co-immunization of pGag-Pol with pVpr. This finding supports that the immune suppressive effect of Vpr is not dependent on any particular antigen. To further examine the specificity of the Vpr effect, we co-immunized mice with pNef and pGag-Pol and pNef and pEnv and assayed again for Nef specific CTL activity. Results indicate that neither pGag-Pol nor pEnv had any effect on Nef-specific CTL activity, indicating that deceased CTL activity was mediated specifically and solely by Vpr. Furthermore, co-immunization of pVpr with pNef or pGag-Pol results in similar inhibition of antigen specific CTL responses suggesting that Vpr as a cell associated/cell free molecule or as a virion-associated molecule mediates the same effect in vivo.

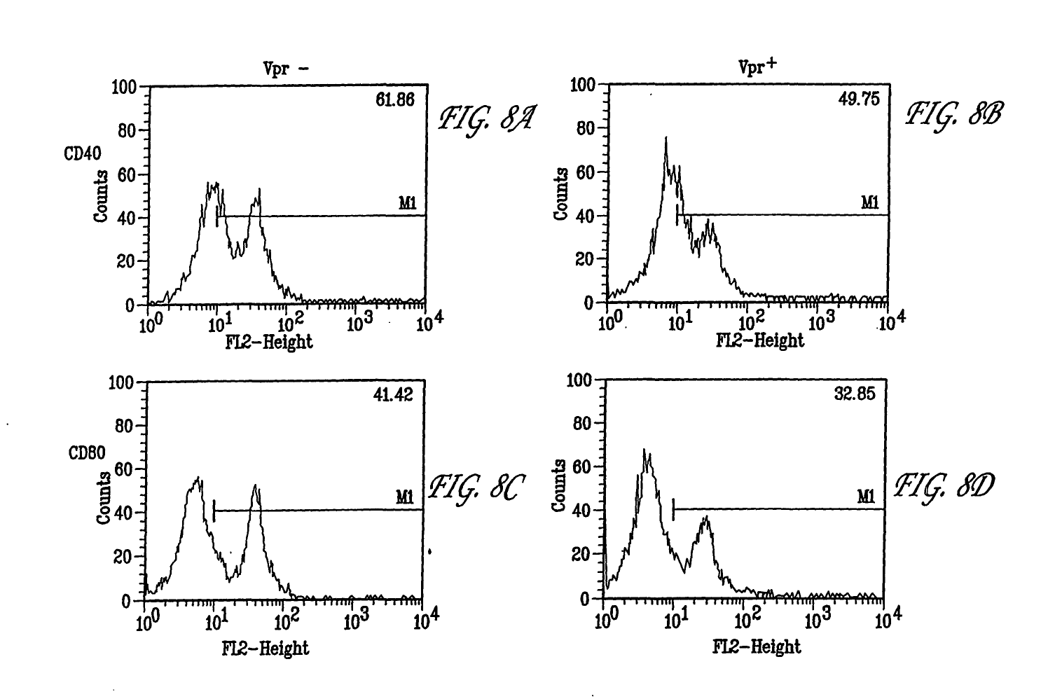

The pattern of cytokine expression influences the nature and persistence of the inflammatory response. For instance, production of IFN-γ and TNFα are well suited to enhance cellular immunity, whereas IL-4 and IL-10 are important for humoral immunity. The in vivo effects of Vpr on the release of the cytokines IL-4 and IFN-γ from antigen stimulated splenocytes collected from immunized mice was examined. FIGS. 6A and 6B show cytokine production in splenocytes obtained from mice co-immunized with pNef in the presence and absence of pVpr. Splenocyctes harvested from mice immunized with pNef with or without pVpr were stimulated with P815 cells infected with vaccinia expressing Nef(vTFnef) for 2 days. Cell-free supernatants were collected and assayed for the production of IL-4 and INF-γ by capture ELISA following the manufacturer's instructions (Intracel, MA). FIGS. 6A and 6B show that splenocytes of mice co-immunized with pVpr and pNef and stimulated with specific antigen produced significantly less IFN-γ compared to mice immunized with pNef and control vector. In contrast, no change was observed in IL-4 production in either group. Mice immunize with pNef in the presence of pVpr produced five fold less IFN-γ (19.9 pg/ml), whereas mice immunized with pNef and control vector produced 95.3 pg/ml of IFN-γ. In parallel with recent in vitro studies that treatment of PBMCs with Vpr suppressed production of certain cytokines (IL-2, IL-12, TNFα), this study provides the first in vivo evidence that the Vpr-mediated immunosuppressive effect is targeted in particular at Th1-mediated cellular immunity.

Effect of Vpr n humoral responses: Since co-immunization of pVpr resulted in down-regulation of CTL responses, the effect of Vpr on humoral responses was also tested by measuring Nef-specific antibodies elicited by pNef immunization in the presence or absence of pVpr by ELISA. FIGS. 7A and 7B show the effect of pVpr on humoral responses generated by different antigens. Mice (n=4) were co-immunized with 100 μg pNef or pPSA and 100 μg of control vector or pVpr intramuscularly at day 0 and boosted on day 14 and again on day 28. The sera samples were collected at 0 and 28 days post immunization and assayed for anti-Nef (FIG. 7A) and anti-PSA (FIG. 7B) specific antibodies at different dilutions. The O.D. values of the pre-immune sera were subtracted from the post-immune sera to account for non-specific binding. These experiments were repeated 3 times with similar results. As shown in FIG. 7A, co-immunization of pVpr with pNef did not alter the Nef-specific antibody titers. Since, pNef by itself does not induce a very high titered antibody response, a plasmid encoding prostate specific antigen (pPSA) which generated a significantly higher humoral response was selected. Mice were immunized with pPSA in the presence of pVpr or control plasmid. The sera from the immunized animals were analyzed for the presence of PSA-specific antibodies by ELISA. The results presented in FIG. 7B show that pPSA alone or in the presence of pVpr induce similar titered antibody responses. The O.D. value for pPSA with vector control or pPSA with pVpr was 0.707 and 0.69, respectively, at a serum dilution of 1:128, which titers accordingly with higher sera dilutions. To reconcile this result with the effects of Vpr on CD8 cell function, antibody subsets as an indicator of the Th1 vs Th2 phenotype were examined. The relative ratios of IgG1 to IgG2a and IgG2a to IgG1 were determined in the presence or absence of pVpr coinjection and are shown in Table 2. The pPSA immunized group had a IgG2a/IgG1 ratio of 0.8. On the other hand, coinjection of pVpr decreased the relative ratio to 0.2, indicating a shift towards a Th2 response. The 4-fold reduction seen in IgG2a/IgG1 ratio with pVpr coinjection indicates that Vpr significantly affects the Th1 type response.

Effect of Vpr on expression of costimulatory molecules of APCs associated with T cell activation: The generation of a T cell immune response is a complex process that requires the engagement of T cells with professional APCs. APCs (including B cells, macrophages and dendritic cells) drive antigen-specific immune responses through the up regulation of CD40, CD80, and CD86 costimulatory molecules as they interact with T cells during initial activation events. To examine Vpr effects on APCs, mouse peritoneal macrophages were infected with VSV-G-Env-complemented HIV-1 vpr⁺ virus or HIV-1 vpr⁻ virus, and the effect of Vpr on costimulatory molecule expression, using an equal number of live cells from both groups was examined. FIGS. 8A, 8B, 8C, and 8D show the effect of Vpr, as a virion-associated molecule, on the expression of costimulatory molecules on antigen presenting cells. Mouse peritoneal macrophages (4×10⁶ cells) were infected with VSV-complemented HIV-1 vpr⁺ virus or HIV-1 vpr⁻virus. Two days post infection equal number of cells (1×10⁶) were gated for GFP and their expression of CD40 and CD80 was determined. Vpr⁺ represents macrophages infected with HIV-1 vpr⁺ virus, and Vpr⁻ represents macrophages infected with HIV-1 vpr⁻ virus. These experiments were repeated 3 times and similar results were obtained. The percentage of cells expressing the respective antigens is shown in each panel. As shown in FIGS. 8A, 8B, 8C, and 8D, the presence of virion-associated Vpr specifically lowered expression of CD40 and CD80 molecules compared to the HIV-1 vpr⁻ virus infected cells. However, expression of CD11b (a macrophage marker) is not affected suggesting that Vpr interferes specifically with costimulatory events. Vpr induced down regulation of these important costimulatory molecules supports that Vpr interferes with early activation events critical for immune induction.

### Discussion

In this study, the role of Vpr, a virion-associated HIV-1 protein, on immune activation was examined. To address the effect of Vpr on host immunity in vivo, we used the DNA vaccination model system. In vivo co-immunization results in multiple plaids being delivered and expressed together in cells in vivo and the expressed foreign antigens are in part taken up by local professional APCs including macrophages and dendritic cells (DCs) through direct transfection mechanisms. Macrophages process the antigen(s) and effectively induce specific immune response to foreign antigens. These studies demonstrate that expression of Vpr can effectively decrease CTL effector function of a co-expressed antigen in vivo. In support it was also observed that Vpr inhibits expression of costimulatory molecules on APCs. These date support that Vpr targets CTL effector function at least in part by interfering with costimulatory molecule expression APCs. Combined with effects on cytokines, these Vpr mediated events would compromise in particular local T cell activation, expansion and T cell survival. Such compromise would be expected to benefit the virus at the ultimate expense of the host. This hypothesis is supported by the data presented here.

Vpr in vivo effects are likely to contribute to an impairment of a localized targeted cellular immune response. Expansion of HIV-1 antigen-specific, CD4⁺ T lymphocytes results in effective maintenance of the immune system and contributes to control of viremia. The presence of a virus-specific, CD8⁺ T cell response is essential for virus clearance in many viral infection models. Additionally, CD8⁺ T cells can inhibit HIV-1 replication in vitro. Recent evidence suggests that CD8⁺ T cells can contribute significantly to control viral load in vivo. The reduction in the number of CD8⁺ T cells in HIV-1 infected patients has been correlated with reduced anti-viral effect and ease progression in parallel with the deterioration of the immune system. In this respect, the data presented here provide in vivo evidence that CD8 effector function is a target of HIV-1 Vpr.

TABLE 1
Comparison of Vpr-mediated cell cycle arrest in human and mouse cells.

| Vpr mutants | HeLa (human) | NIH3T3 (mouse) |
|---|---|---|
| Vpr^{wt} | ++ | ++ |
| αL-A | ++ | ++ |
| E21, 24P | ++ | ++ |
| A30L | − | − |
| A59P | − | − |
| L67S | − | − |
| H71Y | − | − |
| G75A | ++ | ++ |
| C76S | ++ | ++ |
| K95A | − | − |

HeLa and NIH3T3 cells were transfected with 10 μg of different Vpr expression constructs using DOTAP. Transfected cells were selected and analyzed for cell growth and cell cycle arrest by FAGS.

TABLE 2
Effect of Vpr n the relative ratio of IgG1 and IgG2a.

| Plasmids | | | | |
|---|---|---|---|---|
| Used | IgG1 (Th2) | IgG2a (Th1) | IgG1/IgG2a | IgG2a/IgG1 |
| pPSA | 0.461 ± 0.015 | 0.372 ± 0.026 | 1.2 | 0.8 |
| pVpr | 0.006 ± 0.002 | 0.002 ± 0.014 | NA | NA |
| pPSA + | 0.460 ± 0.01 | 0.100 ± 0.002 | 4.6 | 0.2 |
| pVpr | | | | |
| ELISA was performed using PSA-coated (2 μg/ml) 96-well microtiter plates. After blocking the plates for 1 hour with blocking buffer, 50 μg of diluted sera (1:100) were added and incubated for 37° C. for 1 hour. For the determination of relative levels of PSA-specific IgG subclasses, anti-mouse IgG1 and IgG2a conjugated with HRP (Zymed, CA) were substituted for anti-mouse IgG-HRP. This was followed by addition of ABTS (horseradish peroxidase substrate) solution, | | | | |
| #and read at 405 nm using a Dynatec MR5000 plate reader. The relative ratio of IgG1 to IgG2a was calculated as IgG subclass O.D./ total O.D. value. The number of animals used in this experiment to obtain SD was n = 4. Similar results were observed in multiple sets of experiments. NA, not applicable. | | | | |

### Example 3

### Construction of Adenoviral Vector pAdCMV-vpr.

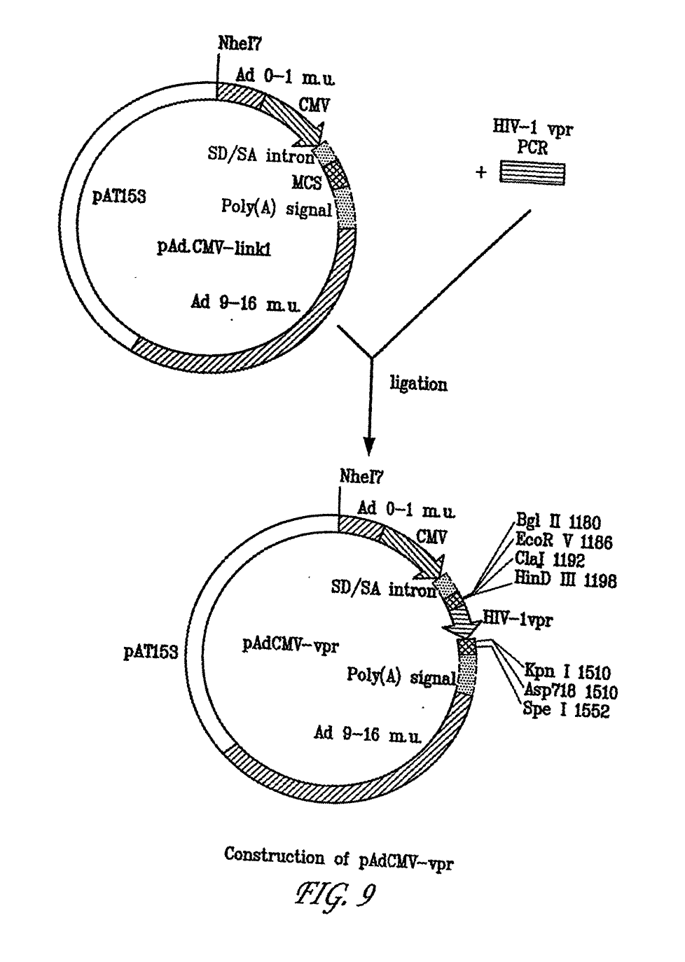

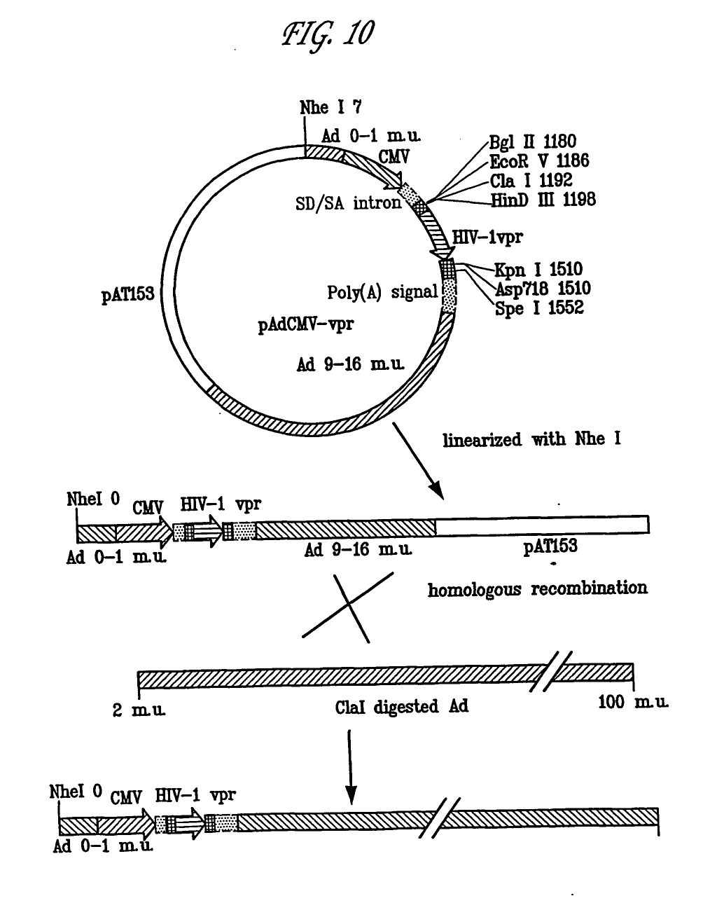

Adenoviral vectors pAdCMV-pr and pAdCMV-lacZ were constructed using pAd.CMV-link1, which is a type 5, E1-deleted, E3-defective adenovirus vector, in which inserted genes are under the transcriptional control of the cytomegaovirus (CMV) promoter (Davis et al., 1998, Gene Therapy, 5:1148-1152, which is incorporated herein by reference). The construction of pAdCMV-lacZ (expressing E. coli β-galactosidase) has been described previously in Davis et al., 1998, supra. The construction of pAdCMV-vpr is described below and represented schematically in FIG. 9.

The proviral construct of HIV-1 stain 89.6 was used as the template, and the primers used were: 5′AAAAGCTTGATGGAACAAGCCCCAGAAGACC 3′ (SEQ D NO:1), containing a HindIII restriction site, and 5′ AATCTAGACTAGGATTTACTGGCTCCATTT 3′ (SEQ D NO:2), containing a XbaI restriction site. The restriction sites are indicated by underlining. The polymerase chain reaction (PCR) cycling conditions were: 94° C. for 4 minutes, 30 cycles of (94° C. for 1 minute, 54° C. for 45 seconds, 72° C. for 30 seconds), and extension at 72° C. for 9 minutes, in a Stratagene-Robocycler Gradient-40 cycler using Taq DNA polymerase (Boehringer Mannheim, Ind.). The PCR product was purified with a PCR purification kit (Qiagen, CA) according to the manufacturer's protocol. The purified PCR product and the pAd.CMV-Link1 vector were digested with restriction enzymes HindIII and XbaI (New England Biolabs, MA), and the respective fragments of 300 bp and 6.7 kb were resolved in and cut from an agarose gel, and purified with a gel purification kit (Qiagen, CA) according to the manufacturer's protocol. Ligation of the two fragments was carried out at a 1:1 molar ratio using T4 DNA ligase (New England Biolabs, MA), followed by transformation into competent bacteria Stbl2 (Life Technologies, MD). Positive clones were identified by restriction enzyme digestion with HindIII and XbaI (Davis et al., supra). The correct, vpr-containing clone was confirmed by DNA sequencing.

### Example 4

### Generation of Adeno-vpr and Adeno-lacZ Recombinant Viral Particles

Recombinant viral particles were generated in 293 cells and purified according to the method described by Davis et al., 1998, supra. The schematic representation of the viral particle generation is presented in FIG. 10. Briefly, the pAdCMV-vpr plasmid was linearized with NheI (New England Biolabs, MA), and resolved in and purified from a low-melting agarose gel. Adenoviral construct sub360 (Davis et al., 1998, supra) was linearized with ClaI (New England Biolabs, ME). The lined pAdCMV-vpr and sub360 were co-transfected into 293 cells using DOTAP transfection agent (Boehringer Mannheim, Ind.). 20 hours after transfection, the cell cultures were overlaid with agar. After 5 days, the cultures were monitored for plaque formations. Plaques were propagated in 293 cells and tested for vpr insertion into the viral genome by PCR and Southern blot analyses. Large quantities of Adeno-vpr virus was purified by the CsCl₂ density gradient centrifugation method. Titers of the stocks were tested by plaque formation in 293 cells with agar overlay. The recombinant adenoviral particle concentration was determined by measuring the optical density at 260 nm, and was expressed as optical particle units (OPU), as described by Mittereder et al., 1996, J. Virol., 70:7498-7509, which is incorporated herein by reference. Adeno-lacZ viral particles were similarly generated and titered

### Example 5

### Transduction (Infection) of Macrophages with Adeno-vpr and Adeno-lacZ Recombinant Viral Particles

Isolation of Human Macrophages

Human PBMCs were isolated from fresh whole blood by density gradient centrifugation with Ficoll-Hypaque (Pharmacia, NJ). The PBMC-containing interface was removed with a Pasteur pipet, and transfered to a new tube, and washed with unsupplemented RPMI medium, three times with centrifugation at 1500 g for 5 minutes. After the final wash, the cells were resuspended, at a concentration of 2×10⁶/ml, in 10% human AB serum in RPMI with 1% penicillin-streptomycin and 1% glutamine. The cells were incubated at 37° C. in polysterene T-75 flasks for 5 days. After the incubation, the cells were washed with RPMI for 3 times to remove non-adherent cells. The adherent monocytes were detached with ethylenediamine tetra-acetic acid (EDTA). The purity of the cell populations, thus isolated, was >98% CD14+, CD3−, as determined by immunofluorescence staining. The cells were incubated in 6-well plates at a density of 1×10⁶ cells/ml in RPMI medium supplemented with 10% human serum, as previously described in Montaner et al., 1997, J. Leukoc. Biol., 62:126-32, which is incorporated herein by reference.

Transduction of Macrophages with Viral Particles

Macrophages were seeded into 6 well tissue culture plates at 2×10⁶ cells per well. Cells were transduced with 50 μl of recombinant virus (either Adeno-vpr or Adeno-lacZ (negative control)) per 5×10⁵ cells (MOI, 2 to 4), in serum-free DMEM medium, and incubated for 90 minutes at 37° C., after which, 1 ml of DMEM with 10% human serum was added to each well. Transduction was confirmed through X-gal detection of β-galactosidase (β-gal) expression from the isogenic control Adeno-lacZ vector. X-gal assays were performed under conditions of low pH to inhibit any endogenous β-gal activity.

### Example 6

### Vpr Protein Expression in Macrophages Transduced with Adeno-vpr Viral Particles

Protein Preparation

Macrophages were infected with recombinant viral particles as described in Example 5. Up to 2×10⁶ cells were collected 48 hours post infection, washed twice in ice-cold PBS, and extracted in 500 μl of lysis buffer containing 50 mM HEPES (H 7.0), 150 mM NaCl, 5 mM EDTA, 0.1% Nonidet P-40, 0.2 mM Pefabloc, 100 mM Na₃VO₄ 10 mM glycerophosphate, 1 mM NaF, and 10 μg/ml (each) of aprotinin, pepstatin, and leupeptin, for 30 minutes at 0° C., after sonication. Cell lysates were centrifuged for 10 min at 0° C. The cell extracts were dialysed against 20 mM Tris (pH 7.5), 5 mM Nacl, 10% glycerol, 0.1 mM EDTA, 1 mM DTT. The final protein concentrations of the extracts were measured using the Bradford reagent (Bio-Rad, CA), using pooled bovine gamma globulin as the standard

Immunob tting Analysis

Immunoblotting (“western”) analyses confirmed that Vpr expression was achieved in cells transduced with Adeno-vpr recombinant viral particles, using an enhanced-chemiluminescence (ECL) detection system (FIG. 11). Immunoblotting was performed after denaturing SDS-PAGE (Laemmli, 1970) of control and Vpr protein from different sources. Briefly, 100 μg of protein extract from cells infected with Adeno-vpr or Aden-lacZ, or 20 μg of purified baculoVpr protein, produced using the BaculoGold baculovirus expression system from Pharmingen (CA) (Muthumani et al., 2000, J. Leukoc. Biol., 68:366-72, which is incorporated herein by reference), were loaded and separated on SDS, 12% polyacrylamide gels. The gels were electroblotted to PVDF membranes (Immobilon P; Millipore, Calif.) and stained with Ponceau Red to control for equal transfer. Filters were blocked in blocking buffer, containing 3% nonfat dry milk and 0.05% Tween 20. After washing, the filters were incubated with anti-Vpr antibodies (1:1,000) in blocking solution overnight at 4° C. After being washed twice, the filters were incubated with a 1:500 dilution of a horseradish peroxidase-conjugated secondary antibody (Boehringer Mannheim, Ind.). After the final washings, immunoreactivity was visualize using the ECL system (Amersham Pharmacia Biotech Ltd., NJ).

### Example 7

### Sub-Cellular Localization of Vpr Protein by Immunofluorescence

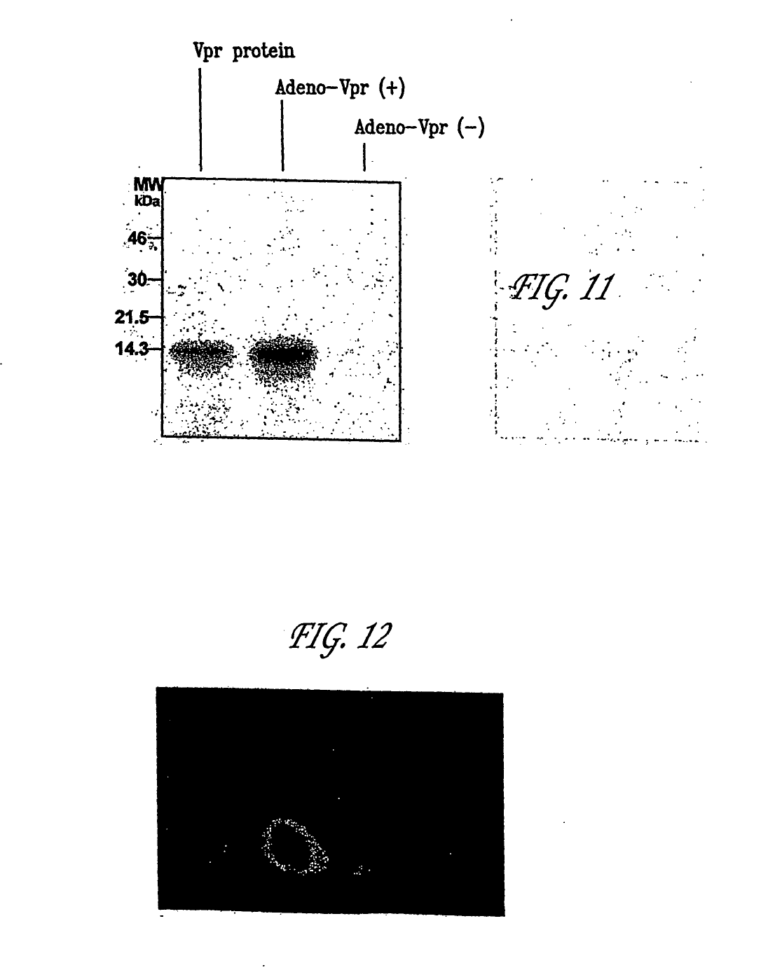

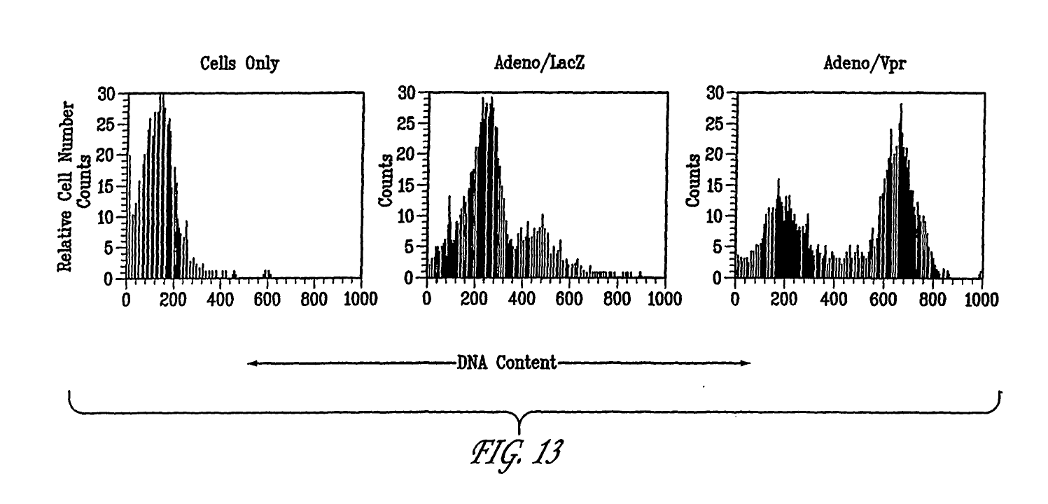

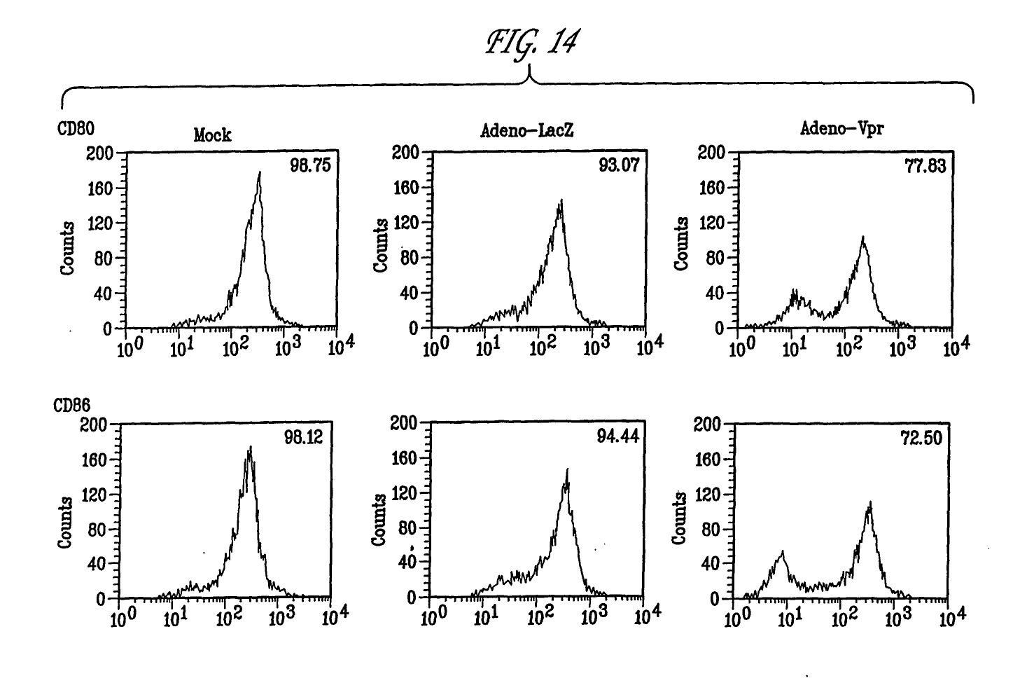

Human macrophages were maintained in DMEM containing 10% human serum and seeded onto poly-L-lysine-coated Falcon glass culture slides (Becton-Dickinson, NJ), at a density of 1×10⁵ cells/ml. Twenty four hours later, the cells were infected with Adeno-vpr viral particles in serum-free medium for 2 hours, after which, the medium was replaced with serum-containing medium. 24 hours post infection, the cells were washed with PBS and fixed with methanol at room temperature for 30 minutes. Adeno-vpr infected cells were fixed and stained with polyclonal anti-Vpr antibody (1:500 dilution), followed by PE-conjugated secondary rabbit antibody staining as described previously (Muthumani et al., 2000, DNA Cell Biol., supra.), stained with DAPI for nuclear staining, and photographed under an immunofluorescence microscope. Vpr expression was seen as red fluorescence in the immunofluorescence photograph in FIG. 12.

### Example 8

### Vpr Protein Enhances G2/M Cell Cycle Arrest

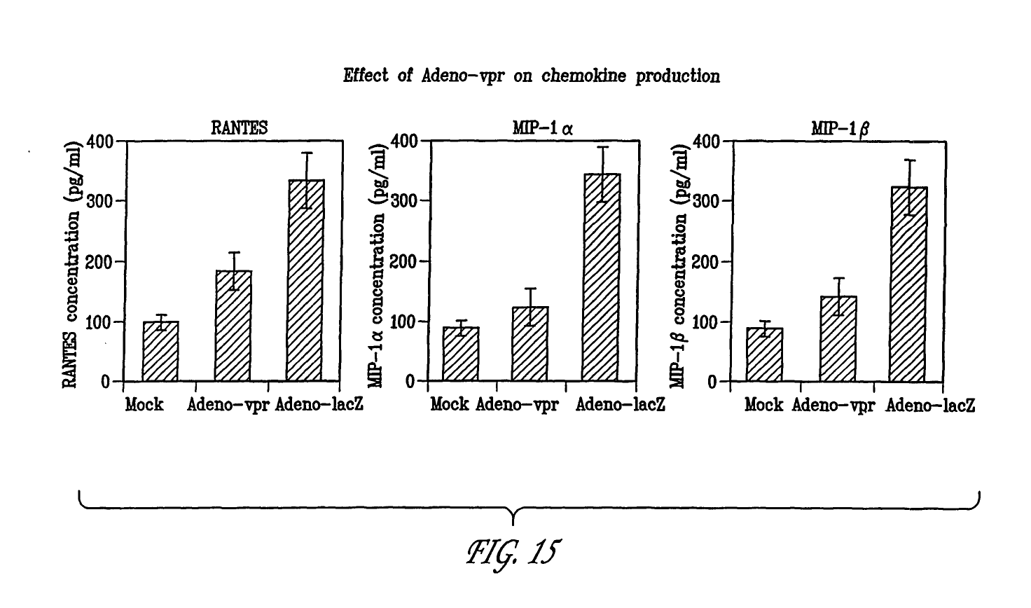

HeLa cells were obtained from ATCC, and were grown in monolayers at 37° C. in 5% CO₂, in DMEM, containing 10% FBS, 1% penicillin, 1% streptomycin, and 1% L-glutamine. HeLa cells (1×10⁶), grown in 35 mm-diameter dishes, were infected with Adeno-vpr and Adeno-lacZ. 48 hours post infection, cells were washed with 2× with PBS, and trypsinized for harvesting. The cells were then incubated with 1 mg/ml RNaseA and 10% propidium iodide in PBS for 30 minutes. Cellular DNA content in the fixed cells was determined with a FACScan flow cytometer and analyzed with the ModFit LT program (Becton Dickinson, CA). Percentage of cells in G2/M was assessed and compared to that of mock infected and Adeno-lacZ infected cells. The results, as presented in FIG. 13, show that the Adeno-lacZ viral particles induce a shift into G2/M phase, and that Vpr expression from Adeno-vpr viral infection enhances the G2/M phase arrest. Similar results were obtained following infection of the human prostate cancer cell line LNCaP (ATCC # CRL-1740), where a 500% increase in G2/M phase cells was effected by infection with Adeno-vpr. Vpr thus enhances the cell growth arresting properties of adenovirus particles.

### Example 9

### Analysis of the Effect of Adeno-vpr Viral Particles on Macrophages by FACS

FACS analysis was performed on infected and mock infected cells to determine if the viral particles affected the expression of macrophage activation markers. Human macrophages were mock infected, or infected with either Adeno-lacZ or Adeno-vpr virus particles, as described in Example 5, above. 48 hours post infection, cells were harvested and single cell suspensions were washed in PBS (pH 7.2), containing 0.2% bovine serum albumin (BSA) and 0.1% NaN₃. Cells were incubated with goat IgG, to block binding of Ig to FcβR, and stained with FITC and PE-conjugated antibodies or IgG control antibody for 60 minutes. Cells were washed with PBS and fixed with 2% paraformaldehyde and analyzed on a fluorescence activated cell sorter (Beckton-Dickinson, CA). FITC-conjugated anti-CD80 and anti-CD86 mAbs, or a PE-conjugated anti-CD14 mAb, purchased from PharMingen (CA) and Coulter (FL). Data were analyzed using the CELL Quest program (Beckton-Dickinson, CA).

The results presented in FIG. 14 show the data for CD80 and CD86 expression. While Adeno-lacZ infection caused a 5.8% and 3.8% decease in CD80 and CD86 expression, respectively, Vpr expression, in the context of Adeno-vpr infection, yielded a 21.2% and 26.1% decrease in CD80 and CD86 expression, respectively. Thus, Vpr expression down-regulates the expression of macrophage activation makers. These data, generated in human macrophages, further support the conclusion that Vpr interferes with costimulatory molecules involved in immune activation.

### Example 10

### Effect of Adeno-vpr Infection on the Secretion of Chemokines by Macrophages

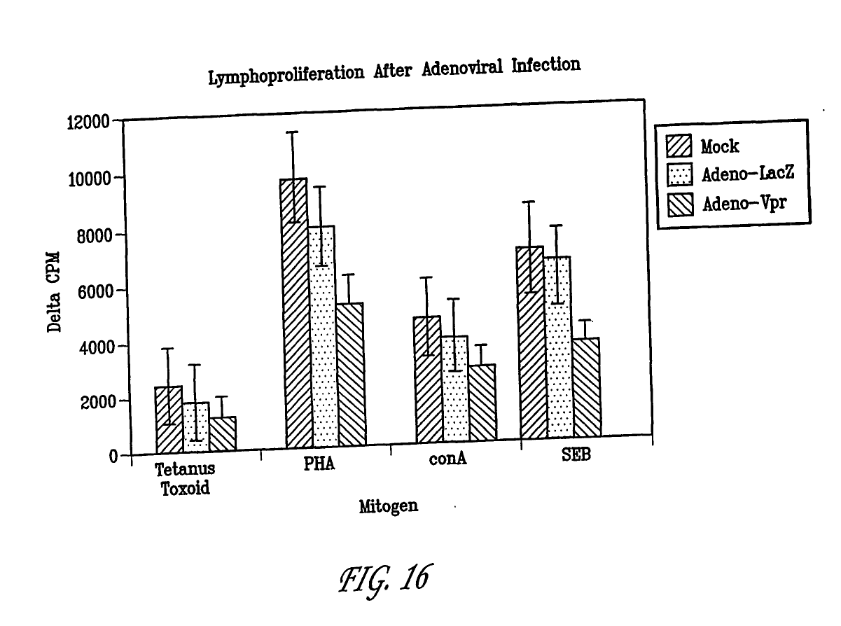

Macrophages were mock infected, or infected with Adeno-vpr or Adeno-lacZ, as described in Example 5. Quantitation of the level of chemokines present in the supernatant was done using a capture ELISA. Specifically, levels of MIP-1α, MIP-1β, and RANTES were measured in the culture supernatants of the infected macrophages. Chemokine assay kits were purchased from R&D Systems (MN), and the assays were performed according to the manufacturer's protocol. Supernatants from the infected cell cultures were added to the wells in triplicate at different dilutions and incubated at 37° C. for 2-3 hours, followed by washing and incubation with detection antibodies for 1 hour. Bound antibodies were developed by the addition of TMB peroxidase substrate and detected at 450 nm in an ELISA plate reader. The results, presented in FIG. 15, reveal that the presence of Vpr protein significantly suppressed the level of chemokine expression induced by adenoviral infection. These results in human macrophages further substantiate the effectiveness of Vpr as an inhibitor of cellular immune responses against gene therapy vectors.

### Example. 11

### Effect of Adeno-vpr Infection on the Lymphoproliferation of PBMCs.

Proliferation assays are used to assess the overall immunocompetence of peripheral blood mononuclear cells, and to detect dividing cells as a function of a test antigen PBMCs were isolated from heparinized blood of normal donors by Ficoll-Hypaque. The isolated cells were suspended at a concentration of 1×10⁶ cells/ml in R10 medium. 100 μl aliquots, containing 1×10⁵ cells, were immediately added to the wells of a 96 well, round-bottom microtiter plate. 100 μl of Adeno-lacZ or Adeno-vpr, at a concentration of 10 viral particles per cell, was added to each well, in triplicate. Mock-, Adeno-lacZ, or Adeno-vpr-infected PBMCs were then separately incubated with the following different mitogenic substances: tetanus toxoid, phytohemagglutinin (PHA), concanavalin-A (ConA), and the superantigen staphylococcal enterotoxin B (SEB). Additionally, to verify the health of the cells, 5 μg/ml of PHA (a non-specific stimulator) was used as a positive control in three wells. Medium alone was used to assess the background level of growth. The cells were incubated at 37° C. in a CO₂ incubator for three days. After three days incubation, 1 μCi of tritiated thymidine was added to each well, followed by overnight (12-18 hours) incubation. The plate was harvested on an automatic 96-well cell harvester, and the amount of incorporated tritiated thymidine was measured in a microbeta counter (Wallace, Turku, Finland), according to Kim et al., 1997, Nat. Biotechnol. July 1997;15(7):641-6, which is incorporated herein by reference. The results are presented in FIG. 16. The values for each condition (Delta CPM) represent the change in incorporated cpm over the baseline incorporated cpm for cells not treated with mitogen. The results, confirm the suppressive effect of Vpr on lymphoproliferation, including the ability of Vpr to suppress lymphoproliferation in response to superantigen SEB.

The foregoing examples are meant to illustrate the invention and are not to be construed to limit the invention in any way. Those skilled in the art will recognize modifications that are within the spirit and scope of the invention. All references cited herein are hereby incorporated by reference in their entirety.

We claim:

1. A method of delivering a desired polypeptide to an individual comprising administer to said individual:
a) an immunogenic vector comprising a nucleic acid encoding the desired polypeptide operably linked to regulatory elements; and
b) one or more of the components selected from the group consisting of:
i) Vpr protein;
ii) a functional fragment of Vpr protein;
iii) a nucleic acid encoding Vpr protein operably linked to regulatory elements; and
iv) a nucleic acid encoding a functional fragment of Vpr protein operably linked to regulatory elements.

2. The method of claim 1 wherein the individual is administered a nucleic acid encoding Vpr protein operably linked to regulatory elements.

3. The method of claim 2 wherein the nucleic acid encoding Vpr protein also encodes the desired polypeptide.

4. The method of claim 2 wherein a nucleic acid encoding Vpr protein and a nucleic acid encoding the desired polypeptide are administered to the individual in the same formulation.

5. The method of claim 4 wherein a nucleic acid encoding Vpr protein and a nucleic acid encoding the desired polypeptide are administered to the individual in separate formulations.

6. The method of claim 1 wherein the individual is administered Vpr protein.

7. The method of claim 6 wherein the Vpr protein and the nucleic acid encoding the desired polypeptide are administered in the same formulation.

8. The method of claim 6 wherein the Vpr protein and the nucleic acid encoding the desired polypeptide are administered in separate formulations.

9. The method of claim 1 wherein the desired polypeptide is a human polypeptide.

10. The method of claim 1 wherein the immunogenic vector is a viral vector.

11. The method of claim 10 wherein the viral vector is an adenoviral vector.

12. A composition comprising an immunogenic vector comprising a nucleic acid encoding the desired polypeptide operably linked to regulatory elements; and one or more of the components selected from the group consisting of:
i) Vpr protein;
ii) a functional fragment of Vpr protein;
iii) a nucleic acid encoding Vpr protein operably linked to regulatory elements; and
iv) a nucleic acid encoding a functional fragment of Vpr protein operably linked to regulatory elements.

13. The composition of claim 12 comprising a nucleic acid encoding Vpr protein operably linked to regulatory elements.

14. The composition of claim 13 comprising a nucleic acid that encodes Vpr protein and the desired polypeptide.

15. The composition of claim 13 comprising Vpr protein.

16. The composition of claim 15 wherein the Vpr protein is incorporated within the immunogenic vector.

17. The composition of claim 15 wherein the immunogenic vector is a viral vector.

18. The composition of claim 17 wherein the viral vector is an adenoviral vector.

19. A method for inhibiting an undesirable immune response in an individual comprising administering to said individual in an amount sufficient to inhibit an undesirable immune response one or more of the components selected from the group consisting of:
i) Vpr protein;
ii) a functional fragment of Vpr protein;
iii) a nucleic acid encoding Vpr protein operably linked to regulatory elements; and
iv) a nucleic acid encoding a functional fragment of Vpr protein operably linked to regulatory elements.

20. The method of claim 19 wherein the individual is administered a nucleic acid encoding Vpr protein operably linked to regulatory elements.

21. The method of claim 19 wherein the individual is administered Vpr protein.

22. The method of claim 19 wherein said individual has an autoimmune/inflammatory disease or condition.

22. The method of claim 19 wherein said individual is undergoing or has undergone a cell, tissue or organ transplant procedure.

23. The method of claim 19 wherein the undesirable immune response is septic shock.

24. The method of claim 23 wherein the component is a prior to the undesirable immune response and the treatment is prophylactic.

25. The method of claim 23 wherein the component is administered during the undesirable immune response and the treatment is therapeutic.

26. The method of claim 19 wherein the undesirable immune response is toxic shock.

27. The method of claim 26 wherein the component is administered prior to the desirable immune response and the treatment is prophylactic.

28. The method of claim 26 wherein the component is administered during the undesirable immune response and the treatment is therapeutic.

29. A method for inhibiting cellular proliferation in a tumor cell in an individual comprising administer to said individual, in an amount sufficient to inhibit cellular proliferation, a recombinant adenovirus comprising a nucleic acid encoding Vpr protein operably linked to regulatory elements or a nucleic acid encoding an anti-tumor fragment of Vpr protein operably linked to regulatory elements.

30. The method of claim 29 wherein the recombinant adenovirus comprises a nucleic acid encoding Vpr protein operably linked to regulatory elements.

31. The method of claim 29 herein the recombinant adenovirus comprises an anti-tumor fragment of Vpr protein operably linked to regulatory elements.

32. The method of claim 29 wherein the recombinant adenovirus is administered by intratumoral injection.
